(19) United States
(12) Patent Application Publication
(10) Pub. No.: US 2025/0170185 A1
(43) Pub. Date: May 29, 2025

(54) AMNIOTIC CYTOKINE FORMULATIONS

(71) Applicant: REGENERATIVE SCIENCE, INC., El Segundo, CA (US)

(72) Inventors: Anthony SAMPIETRO, Hermosa Beach, CA (US); Damien GOLDBERG, Manhattan Beach, CA (US)

(21) Appl. No.: 18/899,892

(22) Filed: Sep. 27, 2024

Related U.S. Application Data
(63) Continuation of application No. 18/166,627, filed on Feb. 9, 2023, now abandoned.
Application No. 18/166,627 is a continuation of application No. PCT/US22/28020, filed on May 6, 2022.
(60) Provisional application No. 63/185,909, filed on May 7, 2021.

(51) Int. Cl. A61K 35/50; A61K 9/00; A61P 27/04

(52) U.S. Cl. CPC A61K 35/50; A61K 9/0048; A61P 27/04

(57) ABSTRACT

Provided herein are compositions and methods for the treatment of ocular surface diseases, such as dry eye syndrome. For example, the disclosure provides formulations, such as amniotic cytokine formulation (ACF) formulations, eye droppers useful for administering the formulations to subjects, and methods of treating subjects using the formulations and/or eye droppers.

### CROSS REFERENCE

This application is a continuation of U.S. patent application Ser. No. 18/166,627 filed Feb. 9, 2023, which is a continuation of International Application No. PCT/US22/28020, filed May 6, 2022, which claims the benefit of U.S. Provisional Patent Application No. 63/185,909, filed May 7, 2021, each of which is incorporated herein by reference in its entirety.

### BACKGROUND

Ocular surface diseases are among the most frequently encountered categories of ocular morbidity in much of the world. Ocular surface diseases can be chronic, and include dry eye syndrome, blepharitis, and other disorders of the cornea. Advancing age, medications, and environmental factors can contribute to the etiology and progression of ocular surface disorders. Thus, therapies that can ameliorate the symptoms of ocular surface disease would be beneficial for a majority of the population.

### INCORPORATION BY REFERENCE

Each patent, publication, and non-patent literature cited in the application is hereby incorporated by reference in its entirety as if each was incorporated by reference individually.

### SUMMARY

Disclosed herein, in some aspects, is a pharmaceutical formulation comprising in a mixture: (a) about 1-10 mg/mL of amniotic membrane; (b) about 10-40% v/v amniotic fluid; and (c) a pharmaceutically-acceptable excipient.

Disclosed herein, in some aspects, is a method of treating a condition in a subject in need thereof, the method comprising: (a) acclimatizing a pharmaceutical formulation at a temperature of at most about 30° C., wherein the pharmaceutical formulation comprises amniotic fluid and a pharmaceutically-acceptable excipient; and (b) administering the pharmaceutical formulation to the subject.

Disclosed herein, in some aspects, is an eye dropper device comprising: (a) a bottle component containing a pharmaceutical formulation that comprises amniotic fluid and a pharmaceutically-acceptable excipient in a mixture; and (b) a nozzle component attached to the bottle component and configured to dispense a dose of the pharmaceutical formulation from the bottle component, wherein the nozzle component comprises a one-way valve that obstructs back-flow of liquid into the bottle component upon dispensing the dose.

Disclosed herein, in some aspects, is a pharmaceutical formulation comprising at least 5 bioactive agents in table 2, each at a concentration of at least 0.1 pg/mL, and a pharmaceutically-acceptable excipient in a mixture.

### BRIEF DESCRIPTION OF THE FIGURES

FIG. 1 shows a National Eye Institute (NEI) conjunctival staining scale for use with Lissamine Green Dye Staining of Conjunctiva.

### DETAILED DESCRIPTION

Ocular surface diseases, such as dry eye syndrome, are among the most frequently encountered categories of ocular morbidity in much of the world. For eye care clinicians, ocular surface diseases remain among the most common reasons for patient visits, and the burden of dry eye syndrome increases as the population ages. For dry eye syndrome patients, the symptoms of chronic ocular discomfort, dryness, and irritation are associated with significant impairment in visual-related quality of life.

Provided herein are compositions and methods for the treatment of ocular surface diseases, such as dry eye syndrome. For example, the disclosure provides formulations, such as amniotic cytokine formulations (ACF), eye droppers useful for administering the formulations to subjects, and methods of treating subjects using the formulations and/or eye droppers.

### Amniotic Cytokine Formulation (ACF) Formulations

The present disclosure provides ACF formulations. ACF formulations can comprise amniotic fluid, amniotic membrane, one or more bioactive agent(s) found in amniotic fluid, one or more bioactive agent(s) found in amniotic membrane, or a combination thereof. ACF formulations can comprise various concentrations of amniotic fluid, amniotic membrane, and/or bioactive agents that are effective for treating a subject with an ocular surface disease, such as dry eye syndrome.

### Amniotic Fluid

An ACF formulation of the disclosure can comprise amniotic fluid. In some embodiments, the amniotic fluid is collected at pregnancy term or at about a full term of pregnancy, for example, at the time of a voluntary cesarean section or vaginal birth. In some embodiments, the amniotic fluid is collected prior to a cesarean section. In some embodiments, the amniotic fluid is collected during a cesarean section. In some embodiments, the amniotic fluid is collected after a cesarean section. In some embodiments, amniotic fluid is collected during pregnancy, for example via amniocentesis. In some embodiments, the amniotic fluid is collected from the second trimester of pregnancy. In some embodiments, the amniotic fluid is collected from the third trimester of pregnancy.

In some embodiments, an ACF of the disclosure comprises at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, or at least about 70% amniotic fluid, for example, volume by volume (v/v), weight by volume (w/v), or weight by weight (w/w). In some embodiments, an ACF of the disclosure comprises at least about 1% v/v amniotic fluid. In some embodiments, an ACF of the disclosure comprises at least about 10% v/v amniotic fluid. In some embodiments, an ACF of the disclosure comprises at least about 15% v/v amniotic fluid. In some embodiments, an ACF of the disclosure comprises at least about 20% v/v amniotic fluid. In some embodiments, an ACF of the disclosure comprises at least about 25% v/v amniotic fluid. In some embodiments, an ACF of the disclosure comprises at least about 30% v/v amniotic fluid.

In some embodiments, an ACF of the disclosure comprises at most about 15%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 70%, or at most about 75% amniotic fluid, for example, volume by volume (v/v), weight by volume (w/v), or weight by weight (w/w). In some embodiments, an ACF of the disclosure comprises at most about 20% v/v amniotic fluid. In some embodiments, an ACF of the disclosure comprises at most about 25% v/v amniotic fluid. In some embodiments, an ACF of the disclosure comprises at most about 30% v/v amniotic fluid. In some embodiments, an ACF of the disclosure comprises at most about 35% v/v amniotic fluid. In some embodiments, an ACF of the disclosure comprises at most about 40% v/v amniotic fluid. In some embodiments, an ACF of the disclosure comprises at most about 50% v/v amniotic fluid.

In some embodiments, an ACF of the disclosure comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, or about 70% amniotic fluid, for example, volume by volume (v/v), weight by volume (w/v), or weight by weight (w/w). In some embodiments, an ACF of the disclosure comprises about 15% v/v amniotic fluid. In some embodiments, an ACF of the disclosure comprises about 20% v/v amniotic fluid. In some embodiments, an ACF of the disclosure comprises about 25% v/v amniotic fluid. In some embodiments, an ACF n of the disclosure comprises about 30% v/v amniotic fluid. In some embodiments, an ACF of the disclosure comprises about 35% v/v amniotic fluid. In some embodiments, an ACF of the disclosure comprises about 40% v/v amniotic fluid.

In some embodiments, an ACF of the disclosure comprises about 1% to about 70%, about 1% to about 60%, about 1% to about 50%, about 1% to about 40%, about 1% to about 35%, about 1% to about 30%, about 1% to about 25%, about 1% to about 20%, about 1% to about 10%, about 5% to about 70%, about 5% to about 60%, about 5% to about 50%, about 5% to about 40%, about 5% to about 35%, about 5% to about 30%, about 5% to about 25%, about 5% to about 20%, about 5% to about 10%, about 10% to about 70%, about 10% to about 60%, about 10% to about 50%, about 10% to about 40%, about 10% to about 35%, about 10% to about 30%, about 10% to about 25%, about 10% to about 20%, about 10% to about 15%, about 15% to about 70%, about 15% to about 60%, about 15% to about 50%, about 15% to about 40%, about 15% to about 35%, about 15% to about 30%, about 15% to about 25%, about 15% to about 20%, about 20% to about 70%, about 20% to about 60%, about 20% to about 50%, about 20% to about 40%, about 20% to about 35%, about 20% to about 30%, about 20% to about 25%, about 25% to about 70%, about 25% to about 60%, about 25% to about 50%, about 25% to about 40%, about 25% to about 35%, about 25% to about 30%, about 30% to about 70%, about 30% to about 60%, about 30% to about 50%, about 30% to about 40%, or about 30% to about 35% amniotic fluid, for example, volume by volume (v/v), weight by volume (w/v), or weight by weight (w/w). In some embodiments, an ACF of the disclosure comprises about 10% to about 40% v/v amniotic fluid. In some embodiments, an ACF of the disclosure comprises about 20% to about 30% v/v amniotic fluid. In some embodiments, an ACF of the disclosure comprises about 20% to about 25% v/v amniotic fluid. In some embodiments, an ACF of the disclosure comprises about 25% to about 30% v/v amniotic fluid.

In some embodiments, an ACF of the disclosure does not contain amniotic fluid. In some embodiments, a formulation disclosed herein contains amniotic membrane and/or contains a set of bioactive agents from non-amniotic fluid sources to approximate the activity of an ACF that contains amniotic fluid.

In some embodiments, an ACF of the disclosure is filtered, for example, through a 0.22 μm or 0.45 μm filter. Filtration can remove, for example, cells, particulates, precipitates, microbial contamination, or a combination thereof. The filter can be configured to not bind or substantially not bind proteins, cytokines, or bioactive agents disclosed herein. In some embodiments, an ACF of the disclosure is cell free or substantially cell free.

### Amniotic Membrane

An ACF of the disclosure can comprise amniotic membrane.

In some embodiments, an ACF of the disclosure comprises at least about 0.001 mg/mL, at least about 0.005 mg/mL, at least about 0.01 mg/mL, at least about 0.05 mg/mL, at least about 0.1 mg/mL, at least about 0.5 mg/mL, at least about 1 mg/mL, at least about 1.5 mg/mL, at least about 2 mg/mL, at least about 2.5 mg/mL, at least about 3 mg/mL, at least about 3.5 mg/mL, at least about 4 mg/mL, at least about 4.5 mg/mL, at least about 5 mg/mL, at least about 5.5 mg/mL, at least about 6 mg/mL, at least about 6.5 mg/mL, at least about 7 mg/mL, at least about 7.5 mg/mL, at least about 8 mg/mL, at least about 8.5 mg/mL, at least about 9 mg/mL, at least about 9.5 mg/mL, at least about 10 mg/mL, at least about 11 mg/mL, at least about 12 mg/mL, at least about 13 mg/mL, at least about 14 mg/mL, at least about 15 mg/mL, at least about 20 mg/mL, at least about 25 mg/mL, at least about 30 mg/mL, at least about 40 mg/mL, at least about 50 mg/mL, or at least about 100 mg/mL of amniotic membrane. In some embodiments, an ACF of the disclosure comprises at least about 1 mg/mL of amniotic membrane. In some embodiments, an ACF of the disclosure comprises at least about 2.5 mg/mL of amniotic membrane. In some embodiments, an ACF of the disclosure comprises at least about 5 mg/mL of amniotic membrane.

In some embodiments, an ACF of the disclosure comprises at most about 0.1 mg/mL, at most about 0.5 mg/mL, at most about 1 mg/mL, at most about 1.5 mg/mL, at most about 2 mg/mL, at most about 2.5 mg/mL, at most about 3 mg/mL, at most about 3.5 mg/mL, at most about 4 mg/mL, at most about 4.5 mg/mL, at most about 5 mg/mL, at most about 5.5 mg/mL, at most about 6 mg/mL, at most about 6.5 mg/mL, at most about 7 mg/mL, at most about 7.5 mg/mL, at most about 8 mg/mL, at most about 8.5 mg/mL, at most about 9 mg/mL, at most about 9.5 mg/mL, at most about 10 mg/mL, at most about 11 mg/mL, at most about 12 mg/mL, at most about 13 mg/mL, at most about 14 mg/mL, at most about 15 mg/mL, at most about 20 mg/mL, at most about 25 mg/mL, at most about 30 mg/mL, at most about 40 mg/mL, at most about 50 mg/mL, most about 100 mg/mL, or at most about 500 mg/ml of amniotic membrane. In some embodiments, an ACF of the disclosure comprises at most about 5 mg/mL of amniotic membrane. In some embodiments, an ACF of the disclosure comprises at most about 7.5 mg/mL of amniotic membrane. In some embodiments, an ACF of the disclosure comprises at most about 10 mg/mL of amniotic membrane.

In some embodiments, an ACF of the disclosure comprises about 0.001 mg/mL, about 0.005 mg/mL, about 0.01 mg/mL, about 0.05 mg/mL, about 0.1 mg/mL, about 0.5 mg/mL, about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, about 10 mg/mL, about 11 mg/mL, about 12 mg/mL, about 13 mg/mL, about 14 mg/mL, about 15 mg/mL, about 20 mg/mL, about 25 mg/mL, about 30 mg/mL, about 40 mg/mL, about 50 mg/mL, or about 100 mg/mL of amniotic membrane. In some embodiments, an ACF of the disclosure comprises about 1 mg/mL of amniotic membrane. In some embodiments, an ACF of the disclosure comprises about 2.5 mg/mL of amniotic membrane. In some embodiments, an ACF of the disclosure comprises about 5 mg/mL of amniotic membrane. In some embodiments, an ACF of the disclosure comprises about 7.5 mg/mL of amniotic membrane. In some embodiments, an ACF of the disclosure comprises about 10 mg/mL of amniotic membrane.

In some embodiments, about an ACF of the disclosure comprises about 0.01 to about 50 mg/mL, about 0.01 to about 25 mg/mL, about 0.01 to about 10 mg/mL, about 0.01 to about 7.5 mg/mL, about 0.01 to about 5 mg/mL, about 0.01 to about 2.5 mg/mL, about 0.01 to about 1 mg/mL, about 0.01 to about 0.5 mg/mL, about 0.1 to about 50 mg/mL, about 0.1 to about 25 mg/mL, about 0.1 to about 10 mg/mL, about 0.1 to about 7.5 mg/mL, about 0.1 to about 5 mg/mL, about 0.1 to about 2.5 mg/mL, about 0.1 to about 1 mg/mL, about 0.1 to about 0.5 mg/mL, about 1 to about 50 mg/mL, about 1 to about 25 mg/mL, about 1 to about 10 mg/mL, about 1 to about 7.5 mg/mL, about 1 to about 5 mg/mL, about 1 to about 2.5 mg/mL, about 2.5 to about 50 mg/mL, about 2.5 to about 25 mg/mL, about 2.5 to about 10 mg/mL, about 2.5 to about 7.5 mg/mL, about 2.5 to about 5 mg/mL, about 5 to about 50 mg/mL, about 5 to about 25 mg/mL, about 5 to about 10 mg/mL, or about 5 to about 7.5 mg/mL of amniotic membrane.

In some embodiments, an ACF of the disclosure comprises at least about 0.001%, at least about 0.002%, at least about 0.003%, at least about 0.004%, at least about 0.005%, at least about 0.006%, at least about 0.007%, at least about 0.008%, at least about 0.009%, at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, or at least about 10% amniotic membrane, for example, weight by volume (w/v) or weight by weight (w/w). In some embodiments, an ACF formulation of the disclosure comprises at least about 0.01% w/v amniotic membrane. In some embodiments, an ACF of the disclosure comprises at least about 0.1% w/v amniotic membrane. In some embodiments, an ACF of the disclosure comprises at least about 0.5% w/v amniotic membrane.

In some embodiments, an ACF of the disclosure comprises at most about 0.01%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 2%, at most about 3%, at most about 4%, at most about 5%, at most about 6%, at most about 7%, at most about 8%, at most about 9%, at most about 10%, at most about 15%, or at most about 20% amniotic membrane, for example, weight by volume (w/v) or weight by weight (w/w). In some embodiments, an ACF of the disclosure comprises at most about 0.5% w/v amniotic membrane. In some embodiments, an ACF of the disclosure comprises at most about 1% w/v amniotic membrane. In some embodiments, an ACF of the disclosure comprises at most about 2% w/v amniotic membrane.

In some embodiments, an ACF of the disclosure comprises about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 15%, or about 20% amniotic membrane, for example, weight by volume (w/v) or weight by weight (w/w). In some embodiments, an ACF of the disclosure comprises about 0.1% w/v amniotic membrane. In some embodiments, an ACF of the disclosure comprises about 0.5% w/v amniotic membrane. In some embodiments, an ACF of the disclosure comprises about 1% w/v amniotic membrane.

In some embodiments, an ACF of the disclosure comprises about 0.001% to about 10%, about 0.001% to about 7.5%, about 0.001% to about 5%, about 0.001% to about 2.5%, about 0.001% to about 1%, about 0.001% to about 0.5%, about 0.001% to about 0.1%, about 0.01% to about 10%, about 0.01% to about 7.5%, about 0.01% to about 5%, about 0.01% to about 2.5%, about 0.01% to about 1%, about 0.01% to about 0.5%, about 0.01% to about 0.1%, about 0.1% to about 10%, about 0.1% to about 7.5%, about 0.1% to about 5%, about 0.1% to about 2.5%, about 0.1% to about 1%, about 0.1% to about 0.5%, about 0.5% to about 10%, about 0.5% to about 7.5%, about 0.5% to about 5%, about 0.5% to about 2.5%, or about 0.5% to about 1% amniotic membrane, for example, weight by volume (w/v) or weight by weight (w/w). In some embodiments, an ACF of the disclosure comprises about 0.01-5% w/v amniotic membrane. In some embodiments, an ACF of the disclosure comprises about 0.1-1% w/v amniotic membrane.

In some embodiments, an ACF of the disclosure does not contain amniotic membrane. In some embodiments, a formulation disclosed herein contains amniotic fluid and/or contains a set of bioactive agents from non-amniotic membrane sources to approximate the activity of an ACF that contains amniotic membrane.

Suitable methods can be utilized to process amniotic membrane to a form suitable for inclusion in a composition or formulation disclosed herein. In some embodiments, amniotic membrane is homogenized by any suitable method, for example, using a tissue homogenizer. In some embodiments, a amniotic membrane (e.g., homogenized amniotic membrane) is filtered to remove debris, particulates, microorganisms, or a combination thereof. In some embodiments, a fluid comprising amniotic membrane is sterile filtered, for example, through a 0.22 μm or 0.45 μm filter. The filter can be configured to not bind or substantially not bind proteins, cytokines, or bioactive agents disclosed herein.

### Bioactive Agents

A composition of the disclosure, such as an ACF, can comprise a bioactive agent. A composition of the disclosure, such as an ACF, can comprise two or more bioactive agents.

In some embodiments, a bioactive agent in a composition of the disclosure is derived from amniotic fluid. In some embodiments, a bioactive agent in a composition of the disclosure is derived from amniotic membrane. In some embodiments, a bioactive agent in a composition of the disclosure is artificially synthesized. In some embodiments, a bioactive agent is removed from a composition to generate a formulation of the disclosure, for example, to reduce an undesired effect the removed bioactive agent has on an ocular surface disease. In some embodiments, two or more, three or more, four or more, or five or more bioactive agents are removed from a composition to generate a formulation of the disclosure.

A bioactive agent can be a protein. A bioactive agent can be, for example, a cytokine, a chemokine, a growth factor, a neurotrophic factor, and epidermal growth factor family member, a TGF-beta superfamily member, a protein kinase, a receptor tyrosine kinase, a hormone, an insulin-like growth factor binding protein family member, an IL-2 cytokine subfamily member, a TIMP family member, a matrix metalloproteinase inhibitor, a TNF superfamily member, a heparin binding protein, or a PDGF/VEGF family member.

Non-limiting examples of cytokines include G-CSF, GM-CSF, HGF, IFN-γ, IL-1α, IL-1β, IL-1rα, IL-2, IL-4, IL-5, IL-6, IL-6R, IL-7, IL-8, IL-10, IL-11, IL-12p40, IL-12p70, IL-13, IL-15, IL-16, IL-17, MCSF, TGFα, TGFβ1, TGFβ2, TGFβ3, TNFα, and TNFβ.

Non-limiting examples of chemokines include BLC (CXCL13), I-309 (CCL1), IL-16, IL-8, MCP-1, MIG (CXCL9), MIP-1α (CCL3), MIP-1β (CCL4), MIP-1δ (CCL15), and RANTES (CCL5).

Non-limiting examples of growth factors include Amphiregulin, BDNF, bFGF, β-NGF, EGF, EG-VEGF, FGF-4, FGF-7, GH, HB-EGF, HGF, IGF-1, PDGF-A, PDGF-B, PDGF-AA, PDGF-BB, PDGF-AB, PLGF, SCF, VEGF, VEGF-A, and VEGF-D.

Non-limiting examples of neurotrophic factors include BDNF, β-NGF, GDNF, NT-3, NT-4, and SCF.

Non-limiting examples of epidermal growth factor family members include Amphiregulin and EGF.

Non-limiting examples of TGF-beta superfamily members include BMP-4, BMP-5, BMP-7, GDF-15, GDNF, TGFβ1, TGFβ2, and TGFβ3.

Non-limiting examples of insulin-like growth factor binding protein family members include IGFBP-1, IGFBP-2, IGFBP-3, IGFBP-4, and IGFBP-6.

Non-limiting examples of matrix metalloproteinase inhibitors include TIMP1 and TIMP2.

In some embodiments, a bioactive agent of the present disclosure can bind to a receptor for example, a cytokine receptor, chemokine receptor, or a growth factor receptor.

In some embodiments, a bioactive agent of the present disclosure modulates (e.g., initiates, increases, decreases, or inhibits) a signaling cascade, for example, ERK signaling, Nanog signaling, NTRK1 signaling, NTRK2 signaling, NGFR signaling, SORCS2 signaling, BCR signaling, EGFR signaling, GPCR signaling, RET signaling, MET signaling, VCAM-1/CD106 signaling, IL-4 signaling, IL-13 signaling, PI3K signaling, AKT signaling, Ras-MAPK signaling, IGFR signaling, JAK-STAT signaling, TGF-beta signaling, calcium signaling, PDGFR signaling, VEGFR signaling, KIT signaling, or a combination thereof.

Non-limiting examples of ERK signaling modulators include Amphiregulin, BDNF, bFGF, BLC (CXCL13), BMP-4, BMP-5, BMP-7, β-NGF, EGFR, EG-VEGF, FGF-4, FGF-7, G-CSF, GDF-15, GH, GM-CSF, HGF, MET, I-309 (CCL1), IGF-1, IL-10, IL-11, IL-12p40, IL-13, IL-15, IL-16, IL-17, IL-1a, IL-1β, IL-2, IL-4, IL-5, IL-6, IL-7, IL-8, MCP-1, MCSF, MIG (CXCL9), MIP-1α (CCL3), MIP-1β (CCL4), MIP-1δ (CCL15), NT-3, PDGF-A, PDGF-B, PDGFAA, PDGF-BB, PDGF-AB, PLGF, RANTES, TGFα, TGFβ1, TGFβ2, TGFβ3, TIMP1, TIMP2, TNFα, TNFβ, and VEGF-D.

Non-limiting examples of Nanog signaling modulators include Amphiregulin, BDNF, BMP-4, BMP-5, BMP-7, GDF-15, GH, NT-3, NT-4, SCF, TIMP1, and TIMP2.

Non-limiting examples of BCR signaling modulators include bFGF, EGF, FGF-4, FGF-7, HB-EGF, HGF, PDGF-A, PDGF-B, PDGFAA, PDGF-BB, PDGF-AB, and SCF.

Non-limiting examples of JAK-STAT signaling modulators include IL-10, IL-11, IL-12, IL-15, and IL-4.

Non-limiting examples of bioactive agents of the disclosure include those listed in TABLE 1. In some embodiments, a composition of the disclosure comprises one or more, two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, or 76 or more of the bioactive agents disclosed in TABLE 1.

In some embodiments, a composition of the disclosure comprises one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, or 77 of the bioactive agents disclosed in TABLE 1.

TABLE 1
TABLE 1: examples of bioactive agents of the disclosure.

| | |
|---|---|
| AR | Amphiregulin |
| BDNF | Brain-derived neurotrophic factor (BDNF) |
| bFGF | Fibroblast growth factor 2 |
| BLC | C-X-C motif chemokine 13/B lymphocyte chemoattractant |
| BMP-4 | Bone morphogenetic protein 4 |
| BMP-5 | Bone morphogenetic protein 5 |
| BMP-7 | Bone morphogenetic protein 7 |
| β-NGF | Beta-nerve growth factor |
| EGF | Pro-epidermal growth factor/Epidermal growth factor/ |
| | Urogastrone |
| EGFR | Epidermal growth factor receptor |
| EG-VEGF | Prokineticin-1/Endocrine-gland-derived vascular endothelial |
| | growth factor |
| FGF-4 | Fibroblast growth factor 4 |
| FGF-7 | Fibroblast growth factor 7 |
| G-CSF | Granulocyte colony-stimulating factor |
| GDF-15 | Growth/differentiation factor 15/Macrophage inhibitory |
| | cytokine 1 (MIC-1) |
| GDNF | Glial cell line-derived neurotrophic factor |
| GH | Somatotropin/Growth hormone |
| GM-CSF | Granulocyte-macrophage colony-stimulating factor |
| HB-EGF | Proheparin-binding EGF-like growth factor/Heparin-binding |
| | EGF-like growth factor |
| HGF | Hepatocyte growth factor |
| I-309 | C-C motif chemokine 1/T lymphocyte-secreted protein I-309 |
| ICAM-1 | Intercellular adhesion molecule 1 |
| IFN-Υ | Interferon gamma |
| IGF-1 | Insulin-like growth factor I |
| IGFBP-1 | Insulin-like growth factor-binding protein 1 |
| IGFBP-2 | Insulin-like growth factor-binding protein 2 |
| IGFBP-3 | Insulin-like growth factor-binding protein 3 |
| IGFBP-4 | Insulin-like growth factor-binding protein 4 |
| IGFBP-6 | Insulin-like growth factor-binding protein 6 |
| IL-10 | Interleukin-10 |
| IL-11 | Interleukin-11 |
| IL-12p40 | Interleukin-12 subunit beta |
| IL-12p70 | Interleukin-12 subunit alpha |
| IL-13 | Interleukin-13 |
| IL-15 | Interleukin-15 |
| IL-16 | Interleukin-16 |
| IL-17 | Interleukin-17A |
| IL-1α | Interleukin-1 alpha |
| IL-1β | Interleukin-1 beta |
| IL-1rα | Interleukin-1 receptor antagonist protein |
| IL-2 | Interleukin-2 |
| IL-4 | Interleukin-4 |
| IL-5 | Interleukin-5 |
| IL-6 | Interleukin-6 |
| IL-6R | Interleukin-6 receptor subunit alpha |
| IL-7 | Interleukin-7 |
| IL-8 | Interleukin-8 |
| MCP-1 | C-C motif chemokine 2/Monocyte chemoattractant protein 1 |
| MCSF | Macrophage colony-stimulating factor 1 |
| MCSFR | Macrophage colony-stimulating factor 1 receptor |
| MIG | C-X-C motif chemokine 9/Monokine induced by interferon- |
| | gamma |
| MIP-1α | C-C motif chemokine 3/Macrophage inflammatory protein 1- |
| | alpha |
| MIP-1β | C-C motif chemokine 4/Macrophage inflammatory protein 1- |
| | beta |
| MIP-1δ | C-C motif chemokine 15/Macrophage inflammatory protein 5 |
| NGFR | Tumor necrosis factor receptor superfamily member 16/Low- |
| | affinity nerve growth factor receptor |
| NT-3 | Neurotrophin-3 |
| NT-4 | Neurotrophin-4 |
| OPG | Tumor necrosis factor receptor superfamily member 11B/ |
| | Osteoprotegerin |
| PDGF-AA | Platelet-derived growth factor subunit A |
| PDGF-BB | Platelet-derived growth factor subunit B |
| PIGF | Placenta growth factor |
| RANTES | C-C motif chemokine 5 |
| SCF | Kit ligand/Stem cell factor |
| SCFR | Mast/stem cell growth factor receptor Kit |
| TGFα | Protransforming growth factor alpha/transforming growth |
| | factor alpha |
| TGFβ1 | Transforming growth factor beta-1 |
| TGFβ3 | Transforming growth factor beta-3 |
| TIMP-1 | Metalloproteinase inhibitor 1/Tissue inhibitor of |
| | metalloproteinases 1 |
| TIMP-2 | Metalloproteinase inhibitor 2/Tissue inhibitor of |
| | metalloproteinases 2 |
| TNFRI | Tumor necrosis factor receptor superfamily member 1A/ |
| | Tumor necrosis factor receptor 1 |
| TNFRII | Tumor necrosis factor receptor superfamily member 1B/ |
| | Tumor necrosis factor receptor 2 |
| TNFα | Tumor necrosis factor/Tumor necrosis factor ligand |
| | superfamily member 2 |
| TNFβ | Lymphotoxin-alpha/Tumor necrosis factor ligand superfamily |
| | member 1 |
| VEGF | Vascular endothelial growth factor A |
| VEGFR2 | Vascular endothelial growth factor receptor 2 |
| VEGFR3 | Vascular endothelial growth factor receptor 3 |
| VEGF-D | Vascular endothelial growth factor D |

In some embodiments, a composition of the disclosure comprises one or more, two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, or all 27 of the bioactive agents in TABLE 2. In some embodiments, a composition of the disclosure comprises 5 or more of the bioactive agents in TABLE 2. In some embodiments, a composition of the disclosure comprises 10 or more of the bioactive agents in TABLE 2. In some embodiments, a composition of the disclosure comprises 15 or more of the bioactive agents in TABLE 2. In some embodiments, a composition of the disclosure comprises 20 or more of the bioactive agents in TABLE 2. In some embodiments, a composition of the disclosure comprises 21 or more of the bioactive agents in TABLE 2. In some embodiments, a composition of the disclosure comprises 22 or more of the bioactive agents in TABLE 2. In some embodiments, a composition of the disclosure comprises 23 or more of the bioactive agents in TABLE 2. In some embodiments, a composition of the disclosure comprises 24 or more of the bioactive agents in TABLE 2. In some embodiments, a composition of the disclosure comprises 25 or more of the bioactive agents in TABLE 2. In some embodiments, a composition of the disclosure comprises 26 or more of the bioactive agents in TABLE 2.

In some embodiments, a composition of the disclosure comprises one, two, three, four, five, six, seven, eight, nine, 10, 11, 12, 13, 14, 15, 16, 17, 18, 19, 20, 21, 22, 23, 24, 25, 26, or all 27 of the bioactive agents in TABLE 2. In some embodiments, a composition of the disclosure comprises 5 of the bioactive agents in TABLE 2. In some embodiments, a composition of the disclosure comprises 6 of the bioactive agents in TABLE 2. In some embodiments, a composition of the disclosure comprises 7 of the bioactive agents in TABLE 2. In some embodiments, a composition of the disclosure comprises 8 of the bioactive agents in TABLE 2. In some embodiments, a composition of the disclosure comprises 9 of the bioactive agents in TABLE 2. In some embodiments, a composition of the disclosure comprises 10 of the bioactive agents in TABLE 2. In some embodiments, a composition of the disclosure comprises 11 of the bioactive agents in TABLE 2. In some embodiments, a composition of the disclosure comprises 12 of the bioactive agents in TABLE 2. In some embodiments, a composition of the disclosure comprises 13 of the bioactive agents in TABLE 2. In some embodiments, a composition of the disclosure comprises 14 of the bioactive agents in TABLE 2. In some embodiments, a composition of the disclosure comprises 15 of the bioactive agents in TABLE 2. In some embodiments, a composition of the disclosure comprises 16 of the bioactive agents in TABLE 2. In some embodiments, a composition of the disclosure comprises 17 of the bioactive agents in TABLE 2. In some embodiments, a composition of the disclosure comprises 18 of the bioactive agents in TABLE 2. In some embodiments, a composition of the disclosure comprises 18 of the bioactive agents in TABLE 2. In some embodiments, a composition of the disclosure comprises 19 of the bioactive agents in TABLE 2. In some embodiments, a composition of the disclosure comprises 20 of the bioactive agents in TABLE 2. In some embodiments, a composition of the disclosure comprises 21 of the bioactive agents in TABLE 2. In some embodiments, a composition of the disclosure comprises 22 of the bioactive agents in TABLE 2. In some embodiments, a composition of the disclosure comprises 23 of the bioactive agents in TABLE 2. In some embodiments, a composition of the disclosure comprises 24 of the bioactive agents in TABLE 2. In some embodiments, a composition of the disclosure comprises 52 of the bioactive agents in TABLE 2. In some embodiments, a composition of the disclosure comprises 26 of the bioactive agents in TABLE 2. In some embodiments, a composition of the disclosure comprises 27 of the bioactive agents in TABLE 2.

TABLE 2
TABLE 2: Examples of bioactive agents of the disclosure.

BLC
EGF
EGF R
EG-VEGF
G-CSF
HGF
ICAM-1
IGFBP-1
IGFBP-2
IGFBP-3
IL-1rα
IL-6
IL-6R
IL-8
MCP-1
MCSF
MCSF R
MIP-1β
MIP-1δ
NGF R
PDGF-AA
PDGF-BB
RANTES
TIMP-1
TIMP-2
TNF RI
TNF RII

A bioactive agent can be present in a composition (e.g., an ACF) of the disclosure at a suitable concentration. In some embodiments, a bioactive agent is present in a composition (e.g., an ACF formulation) at a concentration of at least about 0.001 pg/mL, at least about 0.002 pg/mL, at least about 0.003 pg/mL, at least about 0.004 pg/mL, at least about 0.005 pg/mL, at least about 0.006 pg/mL, at least about 0.007 pg/mL, at least about 0.008 pg/mL, at least about 0.009 pg/mL, at least about 0.01 pg/mL, at least about 0.02 pg/mL, at least about 0.03 pg/mL, at least about 0.04 pg/mL, at least about 0.05 pg/mL, at least about 0.06 pg/mL, at least about 0.07 pg/mL, at least about 0.08 pg/mL, at least about 0.09 pg/mL, at least about 0.1 pg/mL, at least about 2 pg/mL, at least about 0.3 pg/mL, at least about 0.4 pg/mL, at least about 0.5 pg/mL, at least about 0.6 pg/mL, at least about 0.7 pg/mL, at least about 0.8 pg/mL, at least about 0.9 pg/mL, at least about 1 pg/mL, at least about 2 pg/mL, at least about 3 pg/mL, at least about 4 pg/mL, at least about 5 pg/mL, at least about 6 pg/mL, at least about 7 pg/mL, at least about 8 pg/mL, at least about 9 pg/mL, at least about 10 pg/mL, at least about 20 pg/mL, at least about 30 pg/mL, at least about 40 pg/mL, at least about 50 pg/mL, at least about 60 pg/mL, at least about 70 pg/mL, at least about 80 pg/mL, at least about 90 pg/mL, at least about 100 pg/mL, at least about 150 pg/mL, at least about 200 pg/mL, at least about 250 pg/mL, at least about 300 pg/mL, at least about 350 pg/mL, at least about 400 pg/mL, at least about 450 pg/mL, at least about 500 pg/mL, at least about 550 pg/mL, at least about 600 pg/mL, at least about 650 pg/mL, at least about 700 pg/mL, at least about 750 pg/mL, at least about 800 pg/mL, at least about 850 pg/mL, at least about 900 pg/mL, at least about 950 pg/mL, at least about 1000 pg/mL, at least about 1500 pg/mL, at least about 2000 pg/mL, at least about 2500 pg/mL, at least about 3000 pg/mL, at least about 3500 pg/mL, at least about 4000 pg/mL, at least about 4500 pg/mL, at least about 5000 pg/mL, at least about 5500 pg/mL, at least about 6000 pg/mL, at least about 6500 pg/mL, at least about 7000 pg/mL, at least about 8000 pg/mL, at least about 9000 pg/mL, at least about 1×10\hat{ }4 pg/mL, at least about 2×10\hat{ }4 pg/mL, at least about 3×10\hat{ }4 pg/mL, at least about 4×10\hat{ }4 pg/mL, at least about 5×10\hat{ }4 pg/mL, at least about 6×10\hat{ }4 pg/mL, at least about 7×10\hat{ }4 pg/mL, at least about 8×10\hat{ }4 pg/mL, at least about 9×10\hat{ }4 pg/mL, or at least about 1×10\hat{ }5 pg/mL.

In some embodiments, a bioactive agent is present in a composition (e.g., an ACF) at a concentration of at most about 0.001 pg/mL, at most about 0.002 pg/mL, at most about 0.003 pg/mL, at most about 0.004 pg/mL, at most about 0.005 pg/mL, at most about 0.006 pg/mL, at most about 0.007 pg/mL, at most about 0.008 pg/mL, at most about 0.009 pg/mL, at most about 0.01 pg/mL, at most about 0.02 pg/mL, at most about 0.03 pg/mL, at most about 0.04 pg/mL, at most about 0.05 pg/mL, at most about 0.06 pg/mL, at most about 0.07 pg/mL, at most about 0.08 pg/mL, at most about 0.09 pg/mL, at most about 0.1 pg/mL, at most about 0.2 pg/mL, at most about 0.3 pg/mL, at most about 0.4 pg/mL, at most about 0.5 pg/mL, at most about 0.6 pg/mL, at most about 0.7 pg/mL, at most about 0.8 pg/mL, at most about 0.9 pg/mL, at most about 1 pg/mL, at most about 2 pg/mL, at most about 3 pg/mL, at most about 4 pg/mL, at most about 5 pg/mL, at most about 6 pg/mL, at most about 7 pg/mL, at most about 8 pg/mL, at most about 9 pg/mL, at most about 10 pg/mL, at most about 20 pg/mL, at most about 30 pg/mL, at most about 40 pg/mL, at most about 50 pg/mL, at most about 60 pg/mL, at most about 70 pg/mL, at most about 80 pg/mL, at most about 90 pg/mL, at most about 100 pg/mL, at most about 150 pg/mL, at most about 200 pg/mL, at most about 250 pg/mL, at most about 300 pg/mL, at most about 350 pg/mL, at most about 400 pg/mL, at most about 450 pg/mL, at most about 500 pg/mL, at most about 550 pg/mL, at most about 600 pg/mL, at most about 650 pg/mL, at most about 700 pg/mL, at most about 750 pg/mL, at most about 800 pg/mL, at most about 850 pg/mL, at most about 900 pg/mL, at most about 950 pg/mL, at most about 1000 pg/mL, at most about 1500 pg/mL, at most about 2000 pg/mL, at most about 2500 pg/mL, at most about 3000 pg/mL, at most about 3500 pg/mL, at most about 4000 pg/mL, at most about 4500 pg/mL, at most about 5000 pg/mL, at most about 5500 pg/mL, at most about 6000 pg/mL, at most about 6500 pg/mL, at most about 7000 pg/mL, at most about 8000 pg/mL, at most about 9000 pg/mL, at most about 1×10\hat{ }4 pg/mL, at most about 2×10\hat{ }4 pg/mL, at most about 3×10\hat{ }4 pg/mL, at most about 4×10\hat{ }4 pg/mL, at most about 5×10\hat{ }4 pg/mL, at most about 6×10\hat{ }4 pg/mL, at most about 7×10\hat{ }4 pg/mL, at most about 8×10\hat{ }4 pg/mL, at most about 9×10\hat{ }4 pg/mL, or at most about 1×10\hat{ }5 pg/mL.

In some embodiments, a bioactive agent is present in a composition (e.g., an ACF) at a concentration of about 0.001 pg/mL, about 0.002 pg/mL, about 0.003 pg/mL, about 0.004 pg/mL, about 0.005 pg/mL, about 0.006 pg/mL, about 0.007 pg/mL, about 0.008 pg/mL, about 0.009 pg/mL, about 0.01 pg/mL, about 0.02 pg/mL, about 0.03 pg/mL, about 0.04 pg/mL, about 0.05 pg/mL, about 0.06 pg/mL, about 0.07 pg/mL, about 0.08 pg/mL, about 0.09 pg/mL, about 0.1 pg/mL, about 0.2 pg/mL, about 0.3 pg/mL, about 0.4 pg/mL, about 0.5 pg/mL, about 0.6 pg/mL, about 0.7 pg/mL, about 0.8 pg/mL, about 0.9 pg/mL, about 1 pg/mL, about 2 pg/mL, about 3 pg/mL, about 4 pg/mL, about 5 pg/mL, about 6 pg/mL, about 7 pg/mL, about 8 pg/mL, about 9 pg/mL, about 10 pg/mL, about 20 pg/mL, about 30 pg/mL, about 40 pg/mL, about 50 pg/mL, about 60 pg/mL, about 70 pg/mL, about 80 pg/mL, about 90 pg/mL, about 100 pg/mL, about 150 pg/mL, about 200 pg/mL, about 250 pg/mL, about 300 pg/mL, about 350 pg/mL, about 400 pg/mL, about 450 pg/mL, about 500 pg/mL, about 550 pg/mL, about 600 pg/mL, about 650 pg/mL, about 700 pg/mL, about 750 pg/mL, about 800 pg/mL, about 850 pg/mL, about 900 pg/mL, about 950 pg/mL, about 1000 pg/mL, about 1500 pg/mL, about 2000 pg/mL, about 2500 pg/mL, about 3000 pg/mL, about 3500 pg/mL, about 4000 pg/mL, about 4500 pg/mL, about 5000 pg/mL, about 5500 pg/mL, about 6000 pg/mL, about 6500 pg/mL, about 7000 pg/mL, about 8000 pg/mL, about 9000 pg/mL, about 1×10\hat{ }4 pg/mL, about 2×10\hat{ }4 pg/mL, about 3×10\hat{ }4 pg/mL, about 4×10\hat{ }4 pg/mL, about 5×10\hat{ }4 pg/mL, about 6×10\hat{ }4 pg/mL, about 7×10\hat{ }4 pg/mL, about 8×10\hat{ }4 pg/mL, about 9×10\hat{ }4 pg/mL, or about 1×10\hat{ }5 pg/mL.

In some embodiments, a bioactive agent is present in a composition (e.g., an ACF) at a concentration of about 0.001 pg/mL to about 1×10\hat{ }5 pg/mL, about 0.001 pg/mL to about 1×10\hat{ }4 pg/mL, about 0.001 pg/mL to about 1000 pg/mL, about 0.001 pg/mL to about 100 pg/mL, about 0.001 pg/mL to about 10 pg/mL, about 0.001 pg/mL to about 1 pg/mL, about 0.001 pg/mL to about 0.1 pg/mL, about 0.01 pg/mL to about 1×10\hat{ }4 pg/mL, about 0.01 pg/mL to about 1000 pg/mL, about 0.01 pg/mL to about 100 pg/mL, about 0.01 pg/mL to about 10 pg/mL, about 0.01 pg/mL to about 1 pg/mL, about 0.1 pg/mL to about 1×10\hat{ }4 pg/mL, about 0.1 pg/mL to about 1000 pg/mL, about 0.1 pg/mL to about 100 pg/mL, about 0.1 pg/mL to about 10 pg/mL, about 0.1 pg/mL to about 1 pg/mL, about 1 pg/mL to about 1×10\hat{ }4 pg/mL, about 1 pg/mL to about 1000 pg/mL, about 1 pg/mL to about 100 pg/mL, about 1 pg/mL to about 10 pg/mL, about 10 pg/mL to about 1×10\hat{ }4 pg/mL, about 10 pg/mL to about 1000 pg/mL, about 10 pg/mL to about 100 pg/mL, about 1 pg/mL to about 5 pg/mL, about 1 pg/mL to about 50 pg/mL, about 1 pg/mL to about 100 pg/mL, about 1 pg/mL to about 150 pg/mL, about 1 pg/mL to about 200 pg/mL, about 1 pg/mL to about 250 pg/mL, about 1 pg/mL to about 300 pg/mL, about 1 pg/mL to about 350 pg/mL, about 1 pg/mL to about 400 pg/mL, about 1 pg/mL to about 500 pg/mL, about 1 pg/mL to about 600 pg/mL, about 1 pg/mL to about 750 pg/mL, about 1 pg/mL to about 1000 pg/mL, about 10 pg/mL to about 50 pg/mL, about 10 pg/mL to about 100 pg/mL, about 10 pg/mL to about 150 pg/mL, about 10 pg/mL to about 200 pg/mL, about 10 pg/mL to about 250 pg/mL, about 10 pg/mL to about 300 pg/mL, about 10 pg/mL to about 350 pg/mL, about 10 pg/mL to about 400 pg/mL, about 10 pg/mL to about 500 pg/mL, about 10 pg/mL to about 600 pg/mL, about 10 pg/mL to about 750 pg/mL, about 10 pg/mL to about 1000 pg/mL, about 25 pg/mL to about 50 pg/mL, about 25 pg/mL to about 100 pg/mL, about 25 pg/mL to about 150 pg/mL, about 25 pg/mL to about 200 pg/mL, about 25 pg/mL to about 250 pg/mL, about 25 pg/mL to about 300 pg/mL, about 25 pg/mL to about 350 pg/mL, about 25 pg/mL to about 400 pg/mL, about 25 pg/mL to about 500 pg/mL, about 25 pg/mL to about 600 pg/mL, about 25 pg/mL to about 750 pg/mL, about 25 pg/mL to about 1000 pg/mL, about 50 pg/mL to about 100 pg/mL, about 50 pg/mL to about 150 pg/mL, about 50 pg/mL to about 200 pg/mL, about 50 pg/mL to about 250 pg/mL, about 50 pg/mL to about 300 pg/mL, about 50 pg/mL to about 350 pg/mL, about 50 pg/mL to about 400 pg/mL, about 50 pg/mL to about 500 pg/mL, about 50 pg/mL to about 600 pg/mL, about 50 pg/mL to about 750 pg/mL, about 50 pg/mL to about 1000 pg/mL, about 100 pg/mL to about 150 pg/mL, about 100 pg/mL to about 200 pg/mL, about 100 pg/mL to about 250 pg/mL, about 100 pg/mL to about 300 pg/mL, about 100 pg/mL to about 350 pg/mL, about 100 pg/mL to about 400 pg/mL, about 100 pg/mL to about 500 pg/mL, about 100 pg/mL to about 600 pg/mL, about 100 pg/mL to about 750 pg/mL, or about 100 pg/mL to about 1000 pg/mL.

A bioactive agent of the disclosure can be covalently or non-covalently conjugated to another moiety or vehicle. A moiety or vehicle can, for example, provide binding specificity for an additional target, inhibit degradation, increase half-life, increase absorption, reduce toxicity, reduce immunogenicity, and/or increase biological activity of the bioactive agent. Non-limiting examples of the moiety to which the bioactive agent can be conjugated include a Fc domain of an immunoglobulin, a peptide, a lipid, a carbohydrate, a dendrimer, an oligosaccharide, a cholesterol group such as a steroid, and a polymer such as a polyethylene glycol (PEG), a polylysine, or a dextran.

### Pharmaceutical Compositions

A composition of the disclosure, for example, an ACF, can be a pharmaceutical composition that comprises a pharmaceutically-acceptable excipient.

A pharmaceutical composition of the disclosure can be a combination of amniotic fluid, amniotic membrane, and/or any bioactive agents described herein with any other chemical component(s), such as carriers, stabilizers, diluents, dispersing agents, suspending agents, thickening agents, and/or excipients. The pharmaceutical composition can facilitate administration of the amniotic fluid, amniotic membrane, and/or bioactive agents to a subject. Pharmaceutical compositions for administration can include aqueous solutions in water soluble form.

Non-limiting examples of pharmaceutically-acceptable carriers include saline, Ringer's solution, and dextrose solution. Further carriers include sustained release preparations such as semipermeable matrices of solid hydrophobic polymers containing the amniotic fluid, amniotic membrane, and/or bioactive agents. The matrices can be in the form of shaped articles, for example, films, liposomes, microparticles, or microcapsules.

Pharmaceutical formulations can include additional carriers, as well as thickeners, diluents, buffers, preservatives, and surface active agents in addition to the agents disclosed herein.

In some embodiments, a pharmaceutically-acceptable excipient, carrier, or diluent comprises a balanced salt solution (BSS).

A BSS described here can comprise, for example, sodium chloride (NaCl) at about 0.64%, potassium chloride (KCl) at about 0.075%, calcium chloride dihydrate (CaCl₂·2H₂O) at about 0.048%, magnesium chloride hexahydrate (MgCl₂·6H₂O) at about 0.03%, sodium acetate trihydrate (C₂H₃NaO₂·3H₂O) at about 0.39%, sodium citrate dihydrate (C₆H₅Na₃O₇·2H₂O) at about 0.17%, sodium hydroxide and/or hydrochloric acid (to adjust pH), and water. The pH of the BSS can be approximately 7.5. The osmolality of the BSS can be approximately 300 mOsm/Kg.

A BSS described here can comprise, for example, sodium chloride (NaCl) 0.64%, potassium chloride (KCl) 0.075%, calcium chloride dihydrate (CaCl₂)·2H₂O) 0.048%, magnesium chloride hexahydrate (MgCl₂·6H₂O) 0.03%, sodium acetate trihydrate (C₂H₃NaO₂·3H₂O) 0.39%, sodium citrate dihydrate (C₆H₅Na₃O₇·2H₂O) 0.17%, sodium hydroxide and/or hydrochloric acid (to adjust pH), and water. The pH of the BSS can be approximately 7.5. The osmolality of the BSS can be approximately 300 mOsm/Kg.

In some embodiments, a pharmaceutically-acceptable excipient, carrier, or diluent comprises dibasic sodium phosphate. In some embodiments, a pharmaceutically-acceptable excipient, carrier, or diluent comprises sodium bicarbonate. In some embodiments, a pharmaceutically-acceptable excipient, carrier, or diluent comprises dextrose. In some embodiments, a pharmaceutically-acceptable excipient, carrier, or diluent comprises glutathione disulfide (oxidized glutathione).

In some embodiments, a pharmaceutically-acceptable excipient, carrier, or diluent comprises sodium chloride at about 7.14 mg/mL, potassium chloride at about 0.38 mg/mL, calcium chloride dihydrate at about 0.154 mg/mL, magnesium chloride hexahydrate at about 0.2 mg/mL, dibasic sodium phosphate at about 0.42 mg/mL, sodium bicarbonate at about 2.1 mg/mL, dextrose at about 0.92 mg/mL, glutathione disulfide (oxidized glutathione) at about 0.184 mg/mL, hydrochloric acid, and/or sodium hydroxide, in water.

In some embodiments, a pharmaceutically-acceptable excipient, carrier, or diluent comprises a plasmalyte solution. A plasmalyte solution can comprise, for example, sodium chloride (e.g., at about 5.26 g/L), potassium chloride (e.g., at about 0.37 g/L), magnesium chloride hexahydrate (e.g., at about 0.3 g/L), sodium acetate trihydrate (e.g., at about 3.68 g/L), and sodium gluconate (e.g., at about 5.02 g/L).

In some embodiments, the pharmaceutical composition provided herein comprises a therapeutically effective amount of amniotic fluid, amniotic membrane, and/or bioactive agents in admixture with a pharmaceutically-acceptable carrier and/or excipient, for example, saline, phosphate buffered saline, phosphate and amino acids, polymers, polyols, sugar, buffers, preservatives, and other proteins. Illustrative agents include octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene, and glycol. In some embodiments, the pharmaceutical formulation disclosed herein is a stable liquid pharmaceutical formulation.

In some embodiments, suspensions comprising the amniotic fluid, amniotic membrane, and/or bioactive agents can be prepared as oily suspensions. Suitable lipophilic solvents or vehicles include fatty oils such as sesame oil, or synthetic fatty acid esters, such as ethyl oleate or triglycerides, or liposomes. Aqueous suspensions can contain substances which increase the viscosity of the suspension, such as sodium carboxymethyl cellulose, sorbitol, or dextran. The suspension can also contain suitable stabilizers or agents which increase the solubility of bioactive agents therein to allow for the preparation of highly concentrated solutions. In some embodiments, the amniotic fluid, amniotic membrane, and/or bioactive agents can be in prepared in a powder form for constitution with a suitable vehicle, for example, a balanced salt solution or sterile pyrogen-free water, before use.

Pharmaceutical compositions can be formulated using one or more physiologically-acceptable carriers comprising excipients and auxiliaries that facilitate processing of the amniotic fluid, amniotic membrane, and/or bioactive agents into preparations that can be used pharmaceutically. Formulation can be modified depending upon the route of administration chosen. Pharmaceutical compositions comprising bioactive agents described herein can be manufactured, for example, by mixing, dissolving, emulsifying, encapsulating, entrapping, or compression processes.

The pharmaceutical compositions can include a pharmaceutically-acceptable carrier, diluent, or excipient, and amniotic fluid, amniotic membrane, and/or any bioactive agents described herein. Pharmaceutical compositions can contain solubilizers, stabilizers, tonicity enhancing agents, buffers, and preservatives. In some embodiments, a pharmaceutical composition of the disclosure does not contain a preservative, or contains a low concentration of a preservative. In some embodiments, a pharmaceutical composition of the disclosure does not contain Benzalkonium chloride, or contains a low concentration of Benzalkonium chloride. In some embodiments, a pharmaceutical composition of the disclosure does not contain a chemical preservative, or contains a low concentration of a chemical preservative. In some embodiments, a pharmaceutical composition of the disclosure does not contain an oxidative preservative, or contains a low concentration of an oxidative preservative. In some embodiments, the pharmaceutical composition can comprise two or more pharmaceutically-acceptable carriers, diluents, and/or excipients.

Methods for the preparation of compositions comprising the amniotic fluid, amniotic membrane, and/or any bioactive agents described herein include formulating the amniotic fluid, amniotic membrane, and/or bioactive agents with one or more inert, pharmaceutically-acceptable excipients or carriers to form a liquid or semi-solid composition. Liquid compositions include, for example, solutions (e.g., in which the amniotic fluid, amniotic membrane, and/or bioactive agents are dissolved), emulsions comprising amniotic fluid, amniotic membrane, and/or bioactive agents, or a solution containing liposomes, micelles, or nanoparticles comprising amniotic fluid, amniotic membrane, and/or bioactive agents as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the disclosure can be, for example, an immediate release formulation or a controlled release formulation. An immediate release formulation can be formulated to allow the bioactive agents to act rapidly. A controlled release formulation can be a pharmaceutical formulation that has been adapted such that release rates and release profiles of the bioactive agent can be matched to physiological and chronotherapeutic requirements, or has been formulated to effect release of a bioactive agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is in a delayed release form. A delayed release form can be formulated to delay the amniotic fluid, amniotic membrane, and/or bioactive agents action for an extended period of time. A delayed release form can be formulated to delay the release of an effective dose of amniotic fluid, amniotic membrane, and/or bioactive agents, for example, for about 4, about 8, about 12, about 16, or about 24 hours.

A controlled release formulation can be a sustained release form. A sustained release form can be formulated to sustain, for example, the amniotic fluid, amniotic membrane, and/or bioactive agents' action over an extended period of time. A sustained release form can be formulated to provide an effective dose of any amniotic fluid, amniotic membrane, and/or bioactive agents described herein (e.g., provide a physiologically-effective concentration) over a period of about 1, about 2, about 3, about 4, about 6, about 8, about 12, about 16, or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Amniotic fluid, amniotic membrane, and/or bioactive agents described herein can be formulated into pharmaceutical compositions composed of one or more pharmaceutically-acceptable carriers. See e.g., Remington's Pharmaceutical Sciences, latest edition, by E. W. Martin Mack Pub. Co., Easton, PA, incorporated by reference in its entirety, which discloses typical carriers and conventional methods of preparing pharmaceutical compositions. Such carriers can be carriers for administration of compositions to human and non-human subjects, including solutions such as sterile water, saline, and buffered solutions at physiological pH. Pharmaceutical compositions can also include one or more additional active ingredients such as antimicrobial agents, anti-inflammatory agents, and anesthetics.

A pharmaceutical composition disclosed herein can be an ophthalmic formulation. Compositions suitable for topical administration can be used, for example, eye drops. In some embodiments, compositions of the disclosure can comprise a liquid comprising amniotic fluid, amniotic membrane, and/or bioactive agents in solution, in suspension, or both. Liquid compositions can include gels. A liquid composition can be, for example, aqueous.

Aqueous compositions can have ophthalmically-compatible pH and osmolality. The pH of the disclosed composition can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. In some embodiments, the pH of the solution can be from about 5 to about 8, and can be from about 7 to about 7.5. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. A pharmaceutical formulation disclosed herein can have a pH of from about 5.5 to about 6.5. For example, a formulation of the present disclosure can have a pH of about 5.5, about 5.6, about 5.7, about 5.8, about 5.9, about 6.0, about 6.1, about 6.2, about 6.3, about 6.4, or about 6.5. In some embodiments, the pH is 6.2±0.3, 6.2±0.2, 6.2±0.1, about 6.2, or 6.2.

In some embodiments, the pH is 7.5±0.3, 7.5±0.2, 7.5±0.1, about 7.5, or 7.5. In some embodiments, the pH is 7.4±0.3, 7.4±0.2, 7.4±0.1, about 7.4, or 7.4. In some embodiments, the pH is 7.3±0.3, 7.3±0.2, 7.3±0.1, about 7.3, or 7.3. In some embodiments, the pH is 7.2±0.3, 7.2±0.2, 7.2±0.1, about 7.2, or 7.2. In some embodiments, the pH is 7.1±0.3, 7.1±0.2, 7.1±0.1, about 7.1, or 7.1. In some embodiments, the pH is 7±0.3, 7±0.2, 7±0.1, about 7, or 7. In some embodiments, the pH is 6.8±0.3, 6.8±0.2, 6.8±0.1, about 6.8, or 6.8. In some embodiments, the pH is 6.6±0.3, 6.6±0.2, 6.6±0.1, about 6.6, or 6.6. In some embodiments, the pH is 6.4±0.3, 6.4±0.2, 6.4±0.1, about 6.4, or 6.4. In some embodiments, the pH is 6.2±0.3, 6.2±0.2, 6.2±0.1, about 6.2, or 6.2. In some embodiments, the pH is 6±0.3, 6±0.2, 6±0.1, about 6, or 6. If the pH is outside the range desired by the formulator, the pH can be adjusted by using sufficient pharmaceutically-acceptable acids and bases.

In some embodiments, a pharmaceutical formulation or ophthalmic formulation disclosed herein can comprise a buffer. In some embodiments, the buffer serves to maintain a stable pH and to help stabilize the bioactive agents in the formulation. In some embodiments, the buffer or buffer system comprises at least one buffer that has a buffering range that overlaps fully or in part the range of pH 5.5-7.4. In some embodiments, the buffer comprises a sodium phosphate buffer. In some embodiments, the sodium phosphate is present at a concentration of about 5 mM to about 15 mM, about 6 mM to about 14 mM, about 7 mM to about 13 mM, about 8 mM to about 12 mM, about 9 mM to about 11 mM, or about 10 mM.

A composition can be an in situ gellable aqueous composition. In some embodiments, the composition is an in situ gellable aqueous solution. Such a composition can comprise a gelling agent in a concentration effective to promote gelling upon contact with the eye or lacrimal fluid in the exterior of the eye. The composition can comprise an ophthalmic depot formulation comprising an active agent for subconjunctival administration. Microparticles comprising an active agent can be embedded in a biocompatible, pharmaceutically-acceptable polymer or a lipid encapsulating agent. The depot formulations can be adapted to release all or substantially all the active material over an extended period of time. The polymer or lipid matrix, if present, can be adapted to degrade sufficiently to be transported from the site of administration after release of all or substantially all the active agent. The depot formulation can be a liquid formulation, comprising a pharmaceutical acceptable polymer and a dissolved or dispersed active agent. Upon injection, the polymer forms a depot at the injections site, for example, by gelifying or precipitating. The composition can comprise a solid article that can be inserted in a suitable location in the eye, such as between the eye and eyelid or in the conjunctival sac, where the article releases the active agent. Solid articles suitable for implantation in the eye in such fashion can comprise polymers and can be bioerodible or non-bioerodible.

In some embodiments, a pharmaceutical formulation or ophthalmic formulation disclosed herein can comprise a non-ionic detergent. In some embodiments, the non-ionic detergent is a nonionic polymer containing a polyoxyethylene moiety. In some embodiments, the non-ionic detergent is any one or more of polysorbate 20, poloxamer 188 or polyethylene glycol 3350. In some embodiments, the non-ionic detergent is polysorbate 20. In some embodiments, the non-ionic detergent is polysorbate 80.

In some embodiments, a pharmaceutical formulation or ophthalmic formulation disclosed herein can comprise a tonicity agent. In some embodiments, the tonicity agent is sodium chloride or potassium chloride. In some embodiments, the tonicity agent is sodium chloride. In some embodiments, the sodium chloride is present at a concentration of about 5 mM to about 100 mM, about 10 mM to about 50 mM, or about 40 mM.

In some embodiments, a pharmaceutical formulation or ophthalmic formulation disclosed herein can comprise a stabilizer. In some embodiments, the stabilizer is a thermal stabilizer that can stabilize amniotic fluid, amniotic membrane, and/or a bioactive agent disclosed herein under conditions of thermal stress. In some embodiments, the stabilizer maintains at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% of a bioactive agent in a native conformation when the solution containing the bioactive agent and the thermal stabilizer is kept at a suitable storage condition, for example, at about −80° C., at about −20° C., at about 0° C., at about 2° C., at about 4° C., at about 8° C., at about 10° C., at about 15° C., at about 20° C., at about 23° C., at about 25° C., at about 30° C., at about 37° C., or at about 40° C., for at least about 14, at least about 28, at least about 35, at least about 42, at least about 47, at least about 56, at least about 63, at least about 70, or at least about 100 days. In some embodiments, a pharmaceutical formulation of ophthalmic formulation disclosed herein does not contain a stabilizer. In some embodiments, a pharmaceutical formulation of ophthalmic formulation disclosed herein does not contain a thermal stabilizer.

In some embodiments, the stabilizer prevents aggregation of the bioactive agent and at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% of the bioactive agent is not aggregated when the solution containing the bioactive agent and the thermal stabilizer is kept at a suitable storage condition, for example, at about −80° C., at about −20° C., at about 0° C., at about 2° C., at about 4° C., at about 8° C., at about 10° C., at about 15° C., at about 20° C., at about 23° C., at about 25° C., at about 30° C., at about 37° C., at about 40° C., at least about 14, at least about 28, at least about 35, at least about 42, at least about 47, at least about 56, at least about 63, at least about 70, or at least about 100 days.

In some embodiments, the thermal stabilizer is a sugar or sugar alcohol, for example, sucrose, sorbitol, glycerol, trehalose, or mannitol, or any combination thereof. In some embodiments, the stabilizer is a sugar. In some embodiments, the sugar is sucrose, mannitol or trehalose. In some embodiments, the stabilizer is sucrose. In some embodiments, a pharmaceutical formulation or ophthalmic formulation disclosed herein can comprise about 1% to about 20% sugar or sugar alcohol, about 2% to about 18% sugar or sugar alcohol, about 3% to about 15% sugar or sugar alcohol, about 4% to about 10% sugar or sugar alcohol, or about 5% sugar or sugar alcohol. For example, a pharmaceutical formulation or ophthalmic formulation of the present disclosure can comprise about 4%, about 5%, about 6%, about 7%, about 8%, about 9%, about 10%, about 11%, about 12%, about 13%, or about 14% sugar or sugar alcohol (e.g., sucrose, trehalose or mannitol). In some embodiments, the stabilizer is at a concentration of from about 1% w/v to about 20% w/v. In some embodiments, the stabilizer is sucrose at a concentration of from about 1% w/v to about 15% w/v, or from about 1% w/v to about 10% w/v. In some embodiments, the stabilizer is sucrose at a concentration of 5% w/v or about 5% w/v. In some embodiments, the stabilizer is sucrose at a concentration of 7.5% w/v or about 7.5% w/v. In some embodiments, the stabilizer is sucrose at a concentration of 10% w/v or about 10% w/v. In some embodiments, the stabilizer is sucrose at a concentration of 12.5% w/v or about 12.5% w/v. In some embodiments, the stabilizer is sucrose at a concentration of 15% w/v or about 15% w/v. In some embodiments, the stabilizer is sucrose at a concentration of 20% w/v or about 20% w/v.

In some embodiments, the addition of an excipient can change the viscosity of a pharmaceutical composition of the disclosure. In some embodiments the use of an excipient can increase or decrease the viscosity of a fluid by at least 0.001 Pascal-second (Pas), at least 0.0009 Pa's, at least 0.0008 Pas, at least 0.0007 Pa·s, at least 0.0006 Pa's, at least 0.0005 Pa·s, at least 0.0004 Pa·s, at least 0.0003 Pas, at least 0.0002 Pa: s, at least 0.0001 Pa's, at least 0.00005 Pas, or at least 0.00001 Pa's. In some embodiments, viscosity is expressed in centipoise (cP) units, where 1 Pa's is equal to 1000 cP.

In some embodiments, the addition of an excipient to a pharmaceutical composition of the invention can increase or decrease the viscosity of the composition by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, the addition of an excipient to a pharmaceutical composition of the invention can increase or decrease the viscosity of the composition by no greater than 5%, no greater than 10%, no greater than 15%, no greater than 20%, no greater than 25%, no greater than 30%, no greater than 35%, no greater than 40%, no greater than 45%, no greater than 50%, no greater than 55%, no greater than 60%, no greater than 65%, no greater than 70%, no greater than 75%, no greater than 80%, no greater than 85%, no greater than 90%, no greater than 95%, or no greater than 99%.

A pharmaceutical composition described herein can be in a unit dosage form suitable for a single administration of a precise dosage. In unit dosage form, the formulation can be divided into unit doses containing appropriate quantities of one or more compounds, antibodies or therapeutic agents. The unit dosage can be in the form of a package containing discrete quantities of the formulation. Non-limiting examples are packaged single-use eye drop containers, injectables, vials, and ampoules. An aqueous suspension composition disclosed herein can be packaged in a single-dose non-reclosable container. Multiple-dose reclosable containers can be used, for example, in combination with or without a preservative. A formulation for injection disclosed herein can be present in a unit dosage form, for example, in ampoules, or in multi dose containers with a preservative.

### Eye Droppers

In some embodiments, a pharmaceutical formulation (e.g., an ACF) of the disclosure is contained in an eye dropper. The pharmaceutical formulation can be formulated to be dispensed from the eye dropper.

An eye dropper can be a multi-dose eye dropper. In some embodiments, an eye dropper is a single dose eye dropper.

An eye dropper can comprise a bottle component. The bottle component can serve to store the pharmaceutical formulation. The bottle component can be made from or can comprise any suitable material. Non-limiting examples of materials that can be used include high density polyethylene (HDPE), low density polyethylene (LDPE), colorants, anti-microbial agents (e.g., silver ions), and combinations thereof.

In some embodiments an eye dropper contains or has a capacity for at least about 0.1 mL, at least about 0.5 mL, at least about 1 mL, at least about 2 mL, at least about 3 mL, at least about 4 mL, at least about 5 mL, at least about 6 mL, at least about 7 mL, at least about 8 mL, at least about 9 mL, at least about 10 mL, at least about 11 mL, at least about 12 mL, at least about 13 mL, at least about 14 mL, at least about 15 mL, at least about 16 mL, at least about 17 mL, at least about 18 mL, at least about 19 mL, at least about 20 mL, at least about 21 mL, at least about 22 mL, at least about 23 mL, at least about 24 mL, at least about 25 mL, at least about 26 mL, at least about 27 mL, at least about 28 mL, at least about 29 mL, at least about 30 mL, at least about 35 mL, at least about 40 mL, at least about 45 mL, or at least about 50 mL of a pharmaceutical formulation (e.g., an ACF formulation) of the disclosure.

In some embodiments an eye dropper contains or has a capacity for at most about 0.1 mL, at most about 0.5 mL, at most about 1 mL, at most about 2 mL, at most about 3 mL, at most about 4 mL, at most about 5 mL, at most about 6 mL, at most about 7 mL, at most about 8 mL, at most about 9 mL, at most about 10 mL, at most about 11 mL, at most about 12 mL, at most about 13 mL, at most about 14 mL, at most about 15 mL, at most about 16 mL, at most about 17 mL, at most about 18 mL, at most about 19 mL, at most about 20 mL, at most about 21 mL, at most about 22 mL, at most about 23 mL, at most about 24 mL, at most about 25 mL, at most about 26 mL, at most about 27 mL, at most about 28 mL, at most about 29 mL, at most about 30 mL, at most about 35 mL, at most about 40 mL, at most about 45 mL, or at most about 50 mL of a pharmaceutical formulation (e.g., an ACF) of the disclosure.

In some embodiments an eye dropper contains or has a capacity for about 0.1 mL, about 0.5 mL, about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 35 mL, about 40 mL, about 45 mL, or about 50 mL of a pharmaceutical formulation (e.g., an ACF) of the disclosure. In some embodiments an eye dropper contains about 3 mL of a pharmaceutical formulation (e.g., an ACF) of the disclosure. In some embodiments an eye dropper contains about 10 mL of a pharmaceutical formulation (e.g., an ACF) of the disclosure.

In some embodiments an eye dropper contains or has a capacity for about 0.1 mL to about 1 mL, about 0.1 mL to about 2 mL, about 0.1 mL to about 3 mL, about 0.1 mL to about 5 mL, about 0.1 mL to about 7 mL, about 0.1 mL to about 8 mL, about 0.1 mL to about 9 mL, about 0.1 mL to about 10 mL, about 0.1 mL to about 11 mL, about 0.1 mL to about 15 mL, about 0.1 mL to about 20 mL, about 0.1 mL to about 25 mL, about 0.1 mL to about 30 mL, about 0.1 mL to about 50 mL, about 1 mL to about 2 mL, about 1 mL to about 3 mL, about 1 mL to about 5 mL, about 1 mL to about 7 mL, about 1 mL to about 8 mL, about 1 mL to about 9 mL, about 1 mL to about 10 mL, about 1 mL to about 11 mL, about 1 mL to about 15 mL, about 1 mL to about 20 mL, about 1 mL to about 25 mL, about 1 mL to about 30 mL, about 1 mL to about 50 mL, about 3 mL to about 5 mL, about 3 mL to about 7 mL, about 3 mL to about 8 mL, about 3 mL to about 9 mL, about 3 mL to about 10 mL, about 3 mL to about 13 mL, about 3 mL to about 15 mL, about 3 mL to about 20 mL, about 3 mL to about 25 mL, about 3 mL to about 30 mL, about 3 mL to about 50 mL, about 5 mL to about 7 mL, about 5 mL to about 8 mL, about 5 mL to about 9 mL, about 5 mL to about 10 mL, about 5 mL to about 13 mL, about 5 mL to about 15 mL, about 5 mL to about 20 mL, about 5 mL to about 25 mL, about 5 mL to about 30 mL, about 5 mL to about 50 mL, about 7 mL to about 8 mL, about 7 mL to about 9 mL, about 7 mL to about 10 mL, about 7 mL to about 13 mL, about 7 mL to about 15 mL, about 7 mL to about 20 mL, about 7 mL to about 25 mL, about 7 mL to about 30 mL, about 7 mL to about 50 mL, about 10 mL to about 13 mL, about 10 mL to about 15 mL, about 10 mL to about 20 mL, about 10 mL to about 25 mL, about 10 mL to about 30 mL, about 10 mL to about 50 mL, about 15 mL to about 20 mL, about 15 mL to about 25 mL, about 15 mL to about 30 mL, about 15 mL to about 50 mL, about 20 mL to about 25 mL, about 20 mL to about 30 mL, or about 20 mL to about 50 mL of a pharmaceutical formulation (e.g., an ACF) of the disclosure. In some embodiments, an eye dropper of the disclosure comprises about 1 mL to about 15 mL a pharmaceutical formulation (e.g., an ACF) of the disclosure. In some embodiments, an eye dropper of the disclosure comprises about 3 mL to about 15 mL a pharmaceutical formulation (e.g., an ACF) of the disclosure. In some embodiments, an eye dropper of the disclosure comprises about 3 mL to about 10 mL a pharmaceutical formulation (e.g., an ACF) of the disclosure. In some embodiments, an eye dropper of the disclosure comprises about 5 mL to about 15 mL a pharmaceutical formulation (e.g., an ACF) of the disclosure.

A bottle component can have a suitable wall thickness, for example, that allows a dose to be dispensed from the eye dropper upon application of an actuation force disclosed herein, and/or that maintains its integrity upon storage of the eye dropper in a condition disclosed herein (e.g., at a low temperature).

In some embodiments, the bottle component has a wall thickness of at least about 0.01 mm, at least about 0.02 mm, at least about 0.03 mm, at least about 0.04 mm, at least about 0.05 mm, at least about 0.06 mm, at least about 0.07 mm, at least about 0.08 mm, at least about 0.09 mm, at least about 0.1 mm, at least about 0.2 mm, at least about 0.3 mm, at least about 0.4 mm, at least about 0.5 mm, at least about 0.6 mm, at least about 0.7 mm, at least about 0.8 mm, at least about 0.9 mm, at least about 1 mm, at least about 1.1 mm, at least about 1.2 mm, at least about 1.3 mm, at least about 1.4 mm, at least about 1.5 mm, or at least about 2 mm.

In some embodiments, the bottle component has a wall thickness of at most about 0.01 mm, at most about 0.02 mm, at most about 0.03 mm, at most about 0.04 mm, at most about 0.05 mm, at most about 0.06 mm, at most about 0.07 mm, at most about 0.08 mm, at most about 0.09 mm, at most about 0.1 mm, at most about 0.2 mm, at most about 0.3 mm, at most about 0.4 mm, at most about 0.5 mm, at most about 0.6 mm, at most about 0.7 mm, at most about 0.8 mm, at most about 0.9 mm, at most about 1 mm, at most about 1.1 mm, at most about 1.2 mm, at most about 1.3 mm, at most about 1.4 mm, at most about 1.5 mm, or at most about 2 mm.

In some embodiments, the bottle component has a wall thickness of about 0.01 mm, about 0.02 mm, about 0.03 mm, about 0.04 mm, about 0.05 mm, about 0.06 mm, about 0.07 mm, about 0.08 mm, about 0.09 mm, about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, or about 2 mm.

An eye dropper can comprise a nozzle component. A nozzle component can comprise an opening that dispenses a dose of the pharmaceutical formulation, for example, upon application of a suitable actuation force and/or upon inverting the eye dropper such that the opening faces downward.

A nozzle component can comprise any suitable number of sub-components necessary to function as disclosed herein. Non-limiting examples of nozzle sub-components can include a top, a spring, a valve, a plug, a channel, a bottom support, and an outlet orifice. A nozzle component or sub-component can be made from or can comprise any suitable material. Non-limiting examples of materials that can be used include high density polyethylene (HDPE), low density polyethylene (LDPE), colorants, anti-microbial agents (e.g., silver ions), silicones, silicone rubber, and combinations thereof.

A nozzle component can comprise a one-way valve. A one way valve can be used to, for example, reduce or eliminate back-flow of a pharmaceutical formulation into the eye dropper upon dispensing a dose. Reducing back-flow can reduce the likelihood of microbial contamination, reduce the likelihood of particulate contamination, and/or can preserve the purity of the pharmaceutical formulation, for example, allowing for storage of the pharmaceutical formulation in a multi-dose eye dropper without the need for a preservative, or with a reduced concentration of preservative. In some embodiments, reducing a concentration of a preservative in a pharmaceutical (e.g., ACF) formulation of the disclosure can reduce adverse effects of the formulation on the eye, for example, can reduce irritation or inflammation associated with the preservative. In some embodiments, a spring facilitates closure of the one way valve when an actuation force applied to dispense a dose from the eye dropper ceases.

A nozzle component or sub-component thereof (e.g., one-way valve) can comprise an outlet orifice, for example, an orifice through which a dose is dispensed. The outlet orifice can be a suitable diameter to deliver a dose of a pharmaceutical formulation disclosed herein. In some embodiments, the outlet orifice has a diameter of at least about 0.1 mm, at least about 0.2 mm, at least about 0.3 mm, at least about 0.4 mm, at least about 0.5 mm, at least about 0.6 mm, at least about 0.7 mm, at least about 0.8 mm, at least about 0.9 mm, at least about 1 mm, at least about 1.1 mm, at least about 1.2 mm, at least about 1.3 mm, at least about 1.4 mm, at least about 1.5 mm, at least about 1.6 mm, at least about 1.7 mm, at least about 1.8 mm, at least about 1.9 mm, at least about 2 mm, at least about 2.1 mm, at least about 2.2 mm, at least about 2.3 mm, at least about 2.4 mm, at least about 2.5 mm, at least about 2.6 mm, at least about 2.7 mm, at least about 2.8 mm, at least about 2.9 mm, at least about 3 mm, at least about 3.1 mm, at least about 3.2 mm, at least about 3.3 mm, at least about 3.4 mm, at least about 3.5 mm, at least about 3.6 mm, at least about 3.7 mm, at least about 3.8 mm, at least about 3.9 mm, at least about 4 mm, at least about 4.1 mm, at least about 4.2 mm, at least about 4.3 mm, at least about 4.4 mm, at least about 4.5 mm, at least about 4.6 mm, at least about 4.7 mm, at least about 4.8 mm, at least about 4.9 mm, or at least about 5 mm.

In some embodiments, the outlet orifice has a diameter of at most about 0.1 mm, at most about 0.2 mm, at most about 0.3 mm, at most about 0.4 mm, at most about 0.5 mm, at most about 0.6 mm, at most about 0.7 mm, at most about 0.8 mm, at most about 0.9 mm, at most about 1 mm, at most about 1.1 mm, at most about 1.2 mm, at most about 1.3 mm, at most about 1.4 mm, at most about 1.5 mm, at most about 1.6 mm, at most about 1.7 mm, at most about 1.8 mm, at most about 1.9 mm, at most about 2 mm, at most about 2.1 mm, at most about 2.2 mm, at most about 2.3 mm, at most about 2.4 mm, at most about 2.5 mm, at most about 2.6 mm, at most about 2.7 mm, at most about 2.8 mm, at most about 2.9 mm, at most about 3 mm, at most about 3.1 mm, at most about 3.2 mm, at most about 3.3 mm, at most about 3.4 mm, at most about 3.5 mm, at most about 3.6 mm, at most about 3.7 mm, at most about 3.8 mm, at most about 3.9 mm, at most about 4 mm, at most about 4.1 mm, at most about 4.2 mm, at most about 4.3 mm, at most about 4.4 mm, at most about 4.5 mm, at most about 4.6 mm, at most about 4.7 mm, at most about 4.8 mm, at most about 4.9 mm, at most about 5 mm, at most about 5.5 mm at most about 6 mm, at most about 6.5 mm, at most about 7 mm, or at most about 8 mm.

In some embodiments, the outlet orifice has a diameter of about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, or about 5 mm.

In some embodiments, the outlet orifice has a diameter of about 0.1 mm to about 5 mm, of about 0.1 mm to about 4 mm, about 1 mm to about 1.6 mm, about 1 mm to about 1.8 mm, about 1 mm to about 2 mm, about 1 mm to about 2.2 mm, about 1 mm to about 2.4 mm, about 1 mm to about 2.8 mm, about 1 mm to about 3 mm, about 1 mm to about 3.2 mm, about 1 mm to about 3.4 mm, about 1 mm to about 3.6 mm, about 1 mm to about 3.8 mm, about 1 mm to about 4 mm, about 1.4 mm to about 1.6 mm, about 1.4 mm to about 1.8 mm, about 1.4 mm to about 2 mm, about 1.4 mm to about 2.2 mm, about 1.4 mm to about 2.4 mm, about 1.4 mm to about 2.8 mm, about 1.4 mm to about 3 mm, about 1.4 mm to about 3.2 mm, about 1.4 mm to about 3.4 mm, about 1.4 mm to about 3.6 mm, about 1.4 mm to about 3.8 mm, about 1.4 mm to about 4 mm, about 1.6 mm to about 1.8 mm, about 1.6 mm to about 2 mm, about 1.6 mm to about 2.2 mm, about 1.6 mm to about 2.4 mm, about 1.6 mm to about 2.8 mm, about 1.6 mm to about 3 mm, about 1.6 mm to about 3.2 mm, about 1.6 mm to about 3.4 mm, about 1.6 mm to about 3.6 mm, about 1.6 mm to about 3.8 mm, about 1.6 mm to about 4 mm, about 1.8 mm to about 2 mm, about 1.8 mm to about 2.2 mm, about 1.8 mm to about 2.4 mm, about 1.8 mm to about 2.8 mm, about 1.8 mm to about 3 mm, about 1.8 mm to about 3.2 mm, about 1.8 mm to about 3.4 mm, about 1.8 mm to about 3.6 mm, about 1.8 mm to about 3.8 mm, about 1.8 mm to about 4 mm, about 2 mm to about 2.2 mm, about 2 mm to about 2.4 mm, about 2 mm to about 2.8 mm, about 2 mm to about 3 mm, about 2 mm to about 3.2 mm, about 2 mm to about 3.4 mm, about 2 mm to about 3.6 mm, about 2 mm to about 3.8 mm, about 2 mm to about 4 mm, about 2.4 mm to about 2.8 mm, about 2.4 mm to about 3 mm, about 2.4 mm to about 3.2 mm, about 2.4 mm to about 3.4 mm, about 2.4 mm to about 3.6 mm, about 2.4 mm to about 3.8 mm, about 2.4 mm to about 4 mm, about 2.7 mm to about 3 mm, about 2.7 mm to about 3.2 mm, about 2.7 mm to about 3.4 mm, about 2.7 mm to about 3.6 mm, about 2.7 mm to about 3.8 mm, about 2.7 mm to about 4 mm, about 3 mm to about 3.2 mm, about 3 mm to about 3.4 mm, about 3 mm to about 3.6 mm, about 3 mm to about 3.8 mm, or about 3 mm to about 4 mm.

A nozzle component can comprise a semi-permeable membrane. A semi-permeable membrane can facilitate the intake of sterile air into the eye dropper, for example, upon dispensing a dose. In some embodiments, a semi-permeable membrane comprises a pore size that is sufficient to reduce or eliminate passage of microbes and/or other particulates into the eye dropper. In some embodiments, the semi-permeable membrane can reduce the likelihood of microbial contamination, reduce the likelihood of particulate contamination, and/or can preserve the purity of the pharmaceutical formulation, for example, allowing for storage of the pharmaceutical formulation in a multi-dose eye dropper without the need for a preservative, or with a reduced concentration of preservative. A semi-permeable membrane can be made from or can comprise any suitable material or combination thereof. In some embodiments, a semi-permeable membrane is a homogeneous material. In some embodiments, a semi-permeable membrane is silicone rubber. In some embodiments, a semi-permeable membrane is injection-molded silicone rubber. In some embodiments, a semi-permeable membrane of the disclosure does not contain a filter, for example, a 0.22 μm filter. A semi-permeable membrane of the disclosure can have advantageous properties over filters, for example, superior barrier properties can be maintained for a greater duration (e.g., throughout product life), and/or superior performance if wetted.

A nozzle component or sub-component thereof can comprise an antimicrobial agent or treatment, such as silver ions. In some embodiments, an exterior surface of the nozzle component or sub-component thereof comprises an antimicrobial agent or treatment, such as silver ions.

A nozzle component can comprise one or more channels that control the flow rate of liquid. The number and size of the channels can be adjusted to achieve a desired flow rate for a liquid of a viscosity as disclosed herein.

An eye dropper can comprise a cap component, for example, to seal the eye dropper when not in use. In some embodiments, the cap component comprises an antimicrobial agent or treatment, such as silver ions. In some embodiments, a cap component a vent, for example, to promote drying of any residual drop that remains in contact with the outer surface of the nozzle after dispensing a dose. The cap component can be made from or can comprise any suitable material. Non-limiting examples of materials that can be used include high density polyethylene (HDPE), low density polyethylene (LDPE), colorants, anti-microbial agents (e.g., silver ions), and combinations thereof.

An eye dropper of the disclosure can deliver a suitable volume of a pharmaceutical formulation (for example, can be calibrated to deliver a suitable volume of the pharmaceutical formulation per drop dispensed).

In some embodiments, an eye dropper delivers a dose of at least about 5 μL, at least about 10 μL, at least about 15 μL, at least about 16 μL, at least about 17 μL, at least about 18 μL, at least about 19 μL, at least about 20 μL, at least about 21 μL, at least about 22 μL, at least about 23 μL, at least about 24 μL, at least about 25 μL, at least about 26 μL, at least about 27 μL, at least about 28 μL, at least about 29 μL, at least about 30 μL, at least about 31 μL, at least about 32 μL, at least about 33 μL, at least about 34 μL, at least about 35 μL, at least about 36 μL, at least about 37 μL, at least about 38 μL, at least about 39 μL, at least about 40 μL, at least about 41 μL, at least about 42 μL, at least about 43 μL, at least about 44 μL, at least about 45 μL, at least about 46 μL, at least about 47 μL, at least about 48 μL, at least about 49 μL, at least about 50 μL, at least about 60 μL, at least about 70 μL, at least about 80 μL, at least about 90 μL, or at least about 100 μL, for example, per drop dispensed.

In some embodiments, an eye dropper delivers a dose of at most about 10 μL, at most about 15 μL, at most about 16 μL, at most about 17 μL, at most about 18 μL, at most about 19 μL, at most about 20 μL, at most about 21 μL, at most about 22 μL, at most about 23 μL, at most about 24 μL, at most about 25 μL, at most about 26 μL, at most about 27 μL, at most about 28 μL, at most about 29 μL, at most about 30 μL, at most about 31 μL, at most about 32 μL, at most about 33 μL, at most about 34 μL, at most about 35 μL, at most about 36 μL, at most about 37 μL, at most about 38 μL, at most about 39 μL, at most about 40 μL, at most about 41 μL, at most about 42 μL, at most about 43 μL, at most about 44 μL, at most about 45 μL, at most about 46 μL, at most about 47 μL, at most about 48 μL, at most about 49 μL, at most about 50 μL, at most about 60 μL, at most about 70 μL, at most about 80 μL, at most about 90 μL, or at most about 100 μL, for example, per drop dispensed.

In some embodiments, an eye dropper delivers a dose of about 5 μL, about 10 μL, about 15 μL, about 16 μL, about 17 μL, about 18 μL, about 19 μL, about 20 μL, about 21 μL, about 22 μL, about 23 μL, about 24 μL, about 25 μL, about 26 μL, about 27 μL, about 28 μL, about 29 μL, about 30 μL, about 31 μL, about 32 μL, about 33 μL, about 34 μL, about 35 μL, about 36 μL, about 37 μL, about 38 μL, about 39 μL, about 40 μL, about 41 μL, about 42 μL, about 43 μL, about 44 μL, about 45 μL, about 46 μL, about 47 μL, about 48 μL, about 49 μL, about 50 μL, about 60 μL, about 70 μL, about 80 μL, about 90 μL, or about 100 μL, for example, per drop dispensed.

In some embodiments, an eye dropper delivers a dose of about 5 μL to about 20 μL, about 5 μL to about 30 μL, about 5 μL to about 40 μL, about 5 μL to about 50 μL, about 10 μL to about 15 μL, about 10 μL to about 20 μL, about 10 μL to about 22 μL, about 10 μL to about 24 μL, about 10 μL to about 26 μL, about 10 μL to about 28 μL, about 10 μL to about 30 μL, about 10 μL to about 32 μL, about 10 μL to about 34 μL, about 10 μL to about 36 μL, about 10 μL to about 38 μL, about 10 μL to about 40 μL, about 10 μL to about 45 μL, about μL to about 50 μL, about 10 μL to about 60 μL, about 10 μL to about 80 μL, about 10 μL to about 100 μL, about 15 μL to about 20 μL, about 15μL to about 22 μL, about 15 μL to about 24 μL, about 15 μL to about 26 μL, about 15 μL to about 28 μL, about 15 μL to about 30 μL, about 15 μL to about 32 μL, about 15 μL to about 34 μL, about 15 μL to about 36 μL, about 15 μL to about 38 μL, about 15 μL to about 40 μL, about 15 μL to about 45 μL, about μL to about 50 μL, about 15 μL to about 60 μL, about 15 μL to about 80 μL, about 15 μL to about 100 μL, about 20 μL to about 22 μL, about 20μL to about 24 μL, about 20 μL to about 26 μL, about 20 μL to about 28 μL, about 20 μL to about 30 μL, about 20 μL to about 32 μL, about 20 μL to about 34 μL, about 20 μL to about 36 μL, about 20 μL to about 38 μL, about 20 μL to about 40 μL, about 20 μL to about 45 μL, about 20 μL to about 50 μL, about μL to about 60 μL, about 20 μL to about 80 μL, about 20 μL to about 100 μL, about 25 μL to about 26 μL, about 25 μL to about 28 μL, about 25 μL to about 30 μL, about 25 μL to about 32 μL, about 25 μL to about 34 μL, about 25 μL to about 36 μL, about 25 μL to about 38 μL, about 25 μL to about 40 μL, about 25 μL to about 45 μL, about 25 μL to about 50 μL, about 25 μL to about 60 μL, about 25 μL to about 80 μL, about 25 μL to about 100 μL, about 30 μL to about 32 μL, about 30 μL to about 34 μL, about 30 μL to about 36 μL, about 30 μL to about 38 μL, about 30 μL to about 40 μL, about 30 μL to about 45 μL, about 30 μL to about 50 μL, about 30 μL to about 60 μL, about 30 μL to about 80 μL, about 30 μL to about 100 μL, about 35 μL to about 38 μL, about 35 μL to about 40 μL, about 35 μL to about 45 μL, about 35 μL to about 50 μL, about 35 μL to about 60 μL, about 35 μL to about 80 μL, about 35 μL to about 100 μL, about 40 μL to about 45μL, about 40 μL to about 50 μL, about 40 μL to about 60 μL, about 40 μL to about 80 μL, about 40 μL to about 100 μL, about 50 μL to about 60 μL, about 50 μL to about 80 μL, or about 50 μL to about 100 μL, for example, per drop dispensed.

An eye dropper of the disclosure can deliver a dose (e.g., dispense a drop) of a pharmaceutical formulation upon application of a suitable actuation force. An actuation force required for the eye dropper to deliver a dose can be calculated, for example, for the first drop dispensed from the eye dropper, the last drop dispensed from the eye dropper, the median drop dispensed from the eye dropper, or a combination thereof (for example, an average actuation force for all drops delivered from the eyedropper, or a subset thereof).

In some embodiments, an actuation force required for an eye dropper of the disclosure to deliver a dose (e.g., dispense a drop) is at least about 5 N, at least about 10 N, at least about 15 N, at least about 20 N, at least about 25 N, at least about 30 N, at least about 35 N, at least about 40 N, at least about 45 N, at least about 50 N.

In some embodiments, an actuation force required for an eye dropper of the disclosure to deliver a dose (e.g., dispense a drop) is most about 5 N, at most about 10 N, at most about 15 N, at most about 20 N, at most about 25 N, at most about 30 N, at most about 35 N, at most about 40 N, at most about 45 N, at most about 50 N.

In some embodiments, an actuation force required for an eye dropper of the disclosure to deliver a dose (e.g., dispense a drop) is about 5 N, about 10 N, about 15 N, about 20 N, about 25 N, about 30 N, about 35 N, about 40 N, about 45 N, or about 50 N.

In some embodiments, an actuation force required for an eye dropper of the disclosure to deliver a dose (e.g., dispense a dose) is about 5 N to about 10 N, about 5 N to about 15 N, about 5 N to about 20 N, about 5 N to about 25 N, about 5 N to about 30 N, about 5 N to about 35 N, about 5 N to about 40 N, about 5 N to about 50 N, about 10 N to about 15 N, about 10 N to about 20 N, about 10 N to about 25 N, about 10 N to about 30 N, about 10 N to about 35 N, about 10 N to about 40 N, about 10 N to about 50 N, about 15 N to about 20 N, about 15 N to about 25 N, about 15 N to about 30 N, about 15 N to about 35 N, about 15 N to about 40 N, about 15 N to about 50 N, about 20 N to about 25 N, about 20 N to about 30 N, about 20 N to about 35 N, about 20 N to about 40 N, about 20 N to about 50 N, about 25 N to about 30 N, about 25 N to about 35 N, about 25 N to about 40 N, about 25 N to about 50 N, about 30 N to about 35 N, about 30 N to about 40 N, or about 30 N to about 50 N.

### Methods

A therapeutically-effective amount of an ACF described herein can be administered as a pharmaceutical composition to a subject having a disease or condition to be treated. In some embodiments, the subject is a mammal such as a human. A therapeutically-effective amount can vary depending on the severity of the disease, the age and relative health of the subject, the potency of the compounds used, and other factors.

### Subjects and Conditions

Compositions of the disclosure can be used for treating a disease or condition in a subject in need thereof.

Compositions disclosed herein can be sued to treat, for example, ocular surface diseases. Non-limiting examples of ocular surface diseases that can be treated by compositions of the disclosure include dry eye syndrome, mild dry eye syndrome, moderate dry eye syndrome, severe dry eye syndrome, keratoconjunctivitis sicca (KCS), blepharitis, keratitis, conjunctivitis, meibomian gland dysfunction (MDG), allergic eye disease, chemical burns, thermal burns, allergic conjunctivitis (AC), seasonal allergic conjunctivitis (SAC), perennial allergic conjunctivitis (PAC), atopic keratoconjunctivitis (AKC), vernal keratoconjunctivitis (VKC) giant papillary conjunctivitis (GPC), pterygium, Sjogren's Syndrome, chronic punctate keratopathy, filamentary keratopathy, recurrent corneal erosion, bacterial conjunctivitis, culture-negative conjunctivitis, cicatrising (scarring) conjunctivitis, persistent epithelial defect, infectious keratitis, corneal melt, ocular surface failure, rosacea, ocular inflammation, or a combination thereof.

In some embodiments, a subject is 18 years of age or older. In some embodiments, a subject is younger than 18 years of age. A subject can be, for example, an elderly adult, an adult, an adolescent, a pre-adolescent, a child, a toddler, or an infant.

In some embodiments, a subject has a history of keratoconjunctivitis sicca or dry eye syndrome, for example, the subject has reported symptoms or has been diagnosed with the condition for at least 1 week, at least 1 month, at least 3 months, at least 6 months, or at least 1 year. In some embodiments, a subject has a clinical diagnosis of unilateral or bilateral keratoconjunctivitis sicca or Dry Eye Syndrome.

### Dosing and Administration

A pharmaceutical composition disclosed herein can be administered in a therapeutically-effective amount by various routes including, for example, topical administration, parenteral administration, ophthalmic administration, local administration, systemic administration, intraocular administration (e.g., injection), absorption through epithelial or mucocutaneous linings, or any combination thereof.

A pharmaceutical composition disclosed herein can be administered as an eye drop, for example, via an eye dropper and in a drop size disclosed herein. In some embodiments, a dose is one drop, for example, one drop to one eye, or one drop to each eye. In some embodiments, a dose is two drops, for example, two drops to one eye, or two drops to each eye. In some embodiments, a dose is three drops, for example, three drops to one eye, or three drops to each eye. In some embodiments, a dose is four or more drops, for example, four or more drops to one eye, or four or more drops to each eye.

A pharmaceutical composition disclosed herein can be administered to the eye via any suitable form or route including, for example, topical, oral, systemic, intravitreal, intracameral, intracanieral, subconjunctival, subtenon, retrobulbar, intraocular, intrastromal, intracorneal, posterior juxtascleral, periocular, subretinal, or suprachoroidal administration.

A pharmaceutical composition disclosed herein can be delivered via a non-invasive method. Examples of non-invasive modes of administering the formulation can include using a needleless injection device, and topical administration, for example, eye drops to the cornea. A pharmaceutical composition disclosed herein can be administered as an eye drop, for example, via an eye dropper and in a drop size disclosed herein.

The delivery method can include an invasive method for direct delivery of the composition to ocular cells. In some embodiments, a liquid pharmaceutical composition can be delivered via a subretinal injection, intravitreal injection (e.g., front, mid or back vitreal injection), intravitreal implant, intraorbital injection, intraorbital administration, subcutaneous injection, intracameral injection, intracanieral injection, subconjunctival injection, subconjunctival implant, injection into the anterior chamber via the temporal limbus, intrastromal injection, intracorneal injection, aqueous humor injection, subtenon injection, or subtenon implant. The compositions can be administered by injecting the formulation in any part of the eye including anterior chamber, posterior chamber, vitreous chamber (intravitreal), retina proper, and/or subretinal space.

Multiple administration routes can be employed for efficient delivery of the pharmaceutical composition. In some embodiments, the composition is delivered via multiple administration routes, for example, subretinal and topical, to increase the efficiency of delivery.

A pharmaceutical composition disclosed herein can be targeted to any suitable ocular cell including, for example, corneal cells, endothelial cells such as vascular endothelial cells, cells of the retina such as retinal pigment epilthelium (RPE), fibroblasts, astrocytes, glial cells, pericytes, iris epithelial cells, cells of neural origin, ciliary epithelial cells, Müller cells, muscle cells surrounding and attached to the eye such as cells of the lateral rectus muscle, orbital fat cells, cells of the sclera and episclera, cells of the trabecular meshwork, or connective tissue cells.

A pharmaceutical composition disclosed herein can be administered to a subject before, during, or after the occurrence of a disease or condition, and the timing of administering can vary.

The composition can be administered to a subject already suffering from a disease or condition, in an amount sufficient to cure or at least partially arrest the signs and/or symptoms of the disease or condition, or to cure, heal, improve, or ameliorate the condition.

In some embodiments, the composition can be used as a prophylactic and can be administered continuously to subjects with a propensity to conditions or diseases to lessen a likelihood of the occurrence of the disease or condition. In some embodiments, a composition of the disclosure is administered to a subject during or as soon as possible after the onset of the symptoms.

The initial administration can be via any practical route, such as by any route described herein using any formulation described herein.

The composition can be administered for a length of time necessary for the treatment of the disease. The length of treatment can vary for each subject. Length of treatment can vary based on the severity and course of the disease or condition, previous therapy, the subject's health status, weight, and response to the drugs, and the judgment of the treating physician.

Multiple formulations (e.g., ACF) or bioactive agents disclosed herein can be administered in any order or simultaneously. If simultaneously, the multiple formulations or bioactive agents can be provided in a single, unified form, or in multiple forms, for example, as multiple separate eye drops, or as eye drops and injectables. The formulations or bioactive agents can be packed together or separately, in a single package or in a plurality of packages. One or all of the formulations or bioactive agents can be given in multiple doses. If not simultaneous, the timing between the multiple doses can vary, for example, to as much as about a month.

A pharmaceutical composition of the disclosure, for example, a formulation, such as an ACF formulation, can be administered at any interval desired. The administration of the pharmaceutical composition can have regular or irregular dosing schedules to accommodate either the person administering the pharmaceutical composition or the subject receiving the pharmaceutical composition.

In some embodiments, the pharmaceutical composition is administered six times a day, five times a day, four times a day, three times a day, twice a day, once a day, every two days, every three days, every four days, every five days, every six days, five times a week, four times a week, three times a week, two times a week, once a week, once every 10 days, once every two weeks, once every three weeks, once every four weeks, once a month, once every five weeks, once every six weeks, once every eight weeks, once every two months, once every twelve weeks, once every three months, once every four months, once every six months, once a year, or less frequently. In some embodiments, the pharmaceutical composition is administered to the subject as needed (pro re nata; p.r.n.).

In some embodiments, the pharmaceutical composition is administered six times a day. In some embodiments, the pharmaceutical composition is administered five times a day. In some embodiments, the pharmaceutical composition is administered four times a day. In some embodiments, the pharmaceutical composition is administered three times a day. In some embodiments, the pharmaceutical composition is administered two times a day. In some embodiments, the pharmaceutical composition is administered once per day. In some embodiments, the pharmaceutical composition is administered every 2 days. In some embodiments, the pharmaceutical composition is administered every three days.

In some embodiments, the pharmaceutical composition is administered at least six times a day, at least five times a day, at least four times a day, at least three times a day, at least twice a day, at least once a day, at least every two days, at least every three days, at least every four days, at least every five days, at least every six days, at least five times a week, at least four times a week, at least three times a week, at least two times a week, at least once a week, at least once every 10 days, at least once every two weeks, at least once every three weeks, at least once every four weeks, at least once a month, at least once every five weeks, at least once every six weeks, at least once every eight weeks, at least once every two months, at least once every twelve weeks, at least once every three months, at least once every four months, at least once every six months, or at least once a year.

In some embodiments, the pharmaceutical composition is administered at most ten times per day, at most six times a day, at most five times a day, at most four times a day, at most three times a day, at most twice a day, at most once a day, at most every two days, at most every three days, at most every four days, at most every five days, at most every six days, at most five times a week, at most four times a week, at most three times a week, at most two times a week, at most once a week, at most once every 10 days, at most once every two weeks, at most once every three weeks, at most once every four weeks, at most once a month, at most once every five weeks, at most once every six weeks, at most once every eight weeks, at most once every two months, at most once every twelve weeks, at most once every three months, at most once every four months, at most once every six months, or at most once a year.

In some embodiments, the pharmaceutical composition is administered about 1 to about 10 times per day. In some embodiments, the pharmaceutical composition is administered about 1 to about 6 times per day. In some embodiments, the pharmaceutical composition is administered about 1 to about 4 times per day. In some embodiments, the pharmaceutical composition is administered about 1 to about 3 times per day. In some embodiments, the pharmaceutical composition is administered about 1 to about 2 times per day

In some embodiments, the pharmaceutical composition is administered about 2 to about 10 times per day. In some embodiments, the pharmaceutical composition is administered about 2 to about 6 times per day. In some embodiments, the pharmaceutical composition is administered about 2 to about 4 times per day. In some embodiments, the pharmaceutical composition is administered about 2 to about 3 times per day.

The amount administered can be of the same amount in each dose or the dosage can vary between doses. For example, a first amount can be administered in the morning and a second amount can be administered in the evening.

In some embodiments, the pharmaceutical composition is administered to the subject for at least about 1 week, at least about 2 weeks, at least about 3 weeks, at least about 4 weeks, at least about a month, at least about 6 weeks, at least about 8 weeks, at least about 2 months, at least about 3 months, at least about 4 months, at least about 6 months, or at least about a year.

In some embodiments, the pharmaceutical composition is administered to the subject for at most about 1 week, at most about 2 weeks, at most about 3 weeks, at most about 4 weeks, at most about a month, at most about 6 weeks, at most about 8 weeks, at most about 2 months, at most about 3 months, at most about 4 months, at most about 6 months, or at most about a year.

In some embodiments, the pharmaceutical composition is administered to the subject for at about 1 week, at about 2 weeks, at about 3 weeks, at about 4 weeks, at about a month, at about 6 weeks, at about 8 weeks, at about 2 months, at about 3 months, at about 4 months, at about 6 months, or at about a year.

In some embodiments, the pharmaceutical composition is administered to the subject on an ongoing basis.

In some embodiments, a bioactive agent disclosed herein can be administered at a dosage of about 0.0001 mg/kg to about 1000 mg/kg, about 0.001 mg/kg to about 100 mg/kg, about 0.01 mg/kg to about 100 mg/kg, about 0.01 mg/kg to about 20 mg/kg, about 0.02 mg/kg to about 7 mg/kg, about 0.03 mg/kg to about 5 mg/kg, or about 1 mg/kg to about 5 mg/kg by mass of the subject.

Compositions (e.g., ACF formulations) of the disclosure can be acclimatized to a desirable temperature prior to administration to a subject. In some embodiments, acclimatizing the composition as disclosed herein can alter therapeutic properties of the composition upon subsequent administration to the subject, for example, improve the efficacy of a formulation upon administration to the subject.

In some embodiments, a composition (e.g., ACF formulation) of the disclosure is acclimatized to a temperature of at least about −90° C., at least about −80° C., at least about −70° C., at least about −60° C., at least about −50° C., at least about −40° C., at least about −30° C., at least about −20° C., at least about −10° C., at least about −5° C., at least about −2° C., at least about −1° C., at least about 0° C., at least about 1° C., at least about 2° C., at least about 4° C., at least about 6° C., at least about 8° C., at least about 10° C., at least about 12° C., at least about 14° C., at least about 16° C., at least about 18° C., at least about 20° C., at least about 23° C., at least about 25° C., at least about 30° C., at least about 35° C., at least about 37° C., or at least about 40° C. prior to administration to a subject.

In some embodiments, a composition (e.g., ACF formulation) of the disclosure is acclimatized to a temperature of at most about −90° C., at most about −80° C., at most about −70° C., at most about −60° C., at most about −50° C., at most about −40° C., at most about −30° C., at most about −20° C., at most about −10° C., at most about −5° C., at most about −2° C., at most about −1° C., at most about 0° C., at most about 1° C., at most about 2° C., at most about 4° C., at most about 6° C., at most about 8° C., at most about 10° C., at most about 12° C., at most about 14° C., at most about 16° C., at most about 18° C., at most about 20° C., at most about 23° C., at most about 25° C., at most about 30° C., at most about 35° C., at most about 37° C., or at most about 40° C. prior to administration to a subject.

In some embodiments, a composition (e.g., ACF formulation) of the disclosure is acclimatized to a temperature of about −90° C., about −80° C., about −70° C., about −60° C., about −50° C., about −40° C., about −30° C., about −20° C., about −10° C., about −5° C., about −2° C., about −1° C., about 0° C., about 1° C., about 2° C., about 4° C., about 6° C., about 8° C., about 10° C., about 12° C., about 14° C., about 16° C., about 18° C., about 20° C., about 23° C., about 25° C., about 30° C., about 35° C., about 37° C., or about 40° C. prior to administration to a subject.

In some embodiments, a composition (e.g., ACF formulation) of the disclosure is acclimatized to a temperature of about −90° C. to about −80° C., about −90° C. to about −70° C., about −90° C. to about −60° C., about −90° C. to about −50° C., about −90° C. to about −40° C., about −90° C. to about −30° C., about −90° C. to about −20° C., about −90° C. to about −10° C., about −90° C. to about −5° C., about −90° C. to about −2° C., about −90° C. to about −1° C., about −90° C. to about 0° C., about −90° C. to about 1° C., about −90° C. to about 2° C., about −90° C. to about 4° C., about −90° C. to about 6° C., about −90° C. to about 8° C., about −90° C. to about 10° C., about −90° C. to about 12° C., about −90° C. to about 14° C., about −90° C. to about 16° C., about −90° C. to about 18° C., about −90° C. to about 20° C., about −90° C. to about 23° C., about −90° C. to about 25° C., about −90° C. to about 30° C., about −90° C. to about 35° C., about −90° C. to about 37° C., about −90° C. to about 40° C., about −40° C. to about −30° C., about −40° C. to about −20° C., about −40° C. to about −10° C., about −40° C. to about −5° C., about −40° C. to about −2° C., about −40° C. to about −1° C., about −40° C. to about 0° C., about −40° C. to about 1° C., about −40° C. to about 2° C., about −40° C. to about 4° C., about −40° C. to about 6° C., about −40° C. to about 8° C., about −40° C. to about 10° C., about −40° C. to about 12° C., about −40° C. to about 14° C., about −40° C. to about 16° C., about −40° C. to about 18° C., about −40° C. to about 20° C., about −40° C. to about 23° C., about −40° C. to about 25° C., about −40° C. to about 30° C., about −40° C. to about 35° C., about −40° C. to about 37° C., about −40° C. to about 40° C., about −20° C. to about −10° C., about −20° C. to about −5° C., about −20° C. to about −2° C., about −20° C. to about −1° C., about −20° C. to about 0° C., about −20° C. to about 1° C., about −20° C. to about 2° C., about −20° C. to about 4° C., about −20° C. to about 6° C., about −20° C. to about 8° C., about −20° C. to about 10° C., about −20° C. to about 12° C., about −20° C. to about 14° C., about −20° C. to about 16° C., about −20° C. to about 18° C., about −20° C. to about 20° C., about −20° C. to about 23° C., about −20° C. to about 25° C., about −20° C. to about 30° C., about −20° C. to about 35° C., about −20° C. to about 37° C., about −20° C. to about 40° C., about −10° C. to about −5° C., about −10° C. to about −2° C., about −10° C. to about −1° C., about −10° C. to about 0° C., about −10° C. to about 1° C., about −10° C. to about 2° C., about −10° C. to about 4° C., about −10° C. to about 6° C., about −10° C. to about 8° C., about −10° C. to about 10° C., about −10° C. to about 12° C., about −10° C. to about 14° C., about −10° C. to about 16° C., about −10° C. to about 18° C., about −10° C. to about 20° C., about −10° C. to about 23° C., about −10° C. to about 25° C., about −10° C. to about 30° C., about −10° C. to about 35° C., about −10° C. to about 37° C., about −10° C. to about 40° C., about 0° C. to about 0° C., about 0° C. to about 1° C., about 0° C. to about 2° C., about 0° C. to about 4° C., about 0° C. to about 6° C., about 0° C. to about 8° C., about 0° C. to about 10° C., about 0° C. to about 12° C., about 0° C. to about 14° C., about 0° C. to about 16° C., about 0° C. to about 18° C., about 0° C. to about 20° C., about 0° C. to about 23° C., about 0° C. to about 25° C., about 0° C. to about 30° C., about 0° C. to about 35° C., about 0° C. to about 37° C., about 0° C. to about 40° C., about 2° C. to about 4° C., about 2° C. to about 6° C., about 2° C. to about 8° C., about 2° C. to about 10° C., about 2° C. to about 12° C., about 2° C. to about 14° C., about 2° C. to about 16° C., about 2° C. to about 18° C., about 2° C. to about 20° C., about 2° C. to about 23° C., about 2° C. to about 25° C., about 2° C. to about 30° C., about 2° C. to about 35° C., about 2° C. to about 37° C., about 2° C. to about 40° C., about 4° C. to about 6° C., about 4° C. to about 8° C., about 4° C. to about 10° C., about 4° C. to about 12° C., about 4° C. to about 14° C., about 4° C. to about 16° C., about 4° C. to about 18° C., about 4° C. to about 20° C., about 4° C. to about 23° C., about 4° C. to about 25° C., about 4° C. to about 30° C., about 4° C. to about 35° C., about 4° C. to about 37° C., about 4° C. to about 40° C., about 8° C. to about 10° C., about 8° C. to about 12° C., about 8° C. to about 14° C., about 8° C. to about 16° C., about 8° C. to about 18° C., about 8° C. to about 20° C., about 8° C. to about 23° C., about 8° C. to about 25° C., about 8° C. to about 30° C., about 8° C. to about 35° C., about 8° C. to about 37° C., about 8° C. to about 40° C., about 12° C. to about 14° C., about 12° C. to about 16° C., about 12° C. to about 18° C., about 12° C. to about 20° C., about 12° C. to about 23° C., about 12° C. to about 25° C., about 12° C. to about 30° C., about 12° C. to about 35° C., about 12° C. to about 37° C., about 12° C. to about 40° C., about 16° C. to about 18° C., about 16° C. to about 20° C., about 16° C. to about 23° C., about 16° C. to about 25° C., about 16° C. to about 30° C., about 16° C. to about 35° C., about 16° C. to about 37° C., about 12° C. to about 40° C., about 20° C. to about 23° C., about 20° C. to about 25° C., about 20° C. to about 30° C., about 20° C. to about 35° C., about 20° C. to about 37° C., about 20° C. to about 40° C., about 23° C. to about 25° C., about 23° C. to about 30° C., about 23° C. to about 35° C., about 23° C. to about 37° C., about 23° C. to about 40° C., about 25° C. to about 30° C., about 25° C. to about 35° C., about 25° C. to about 37° C., about 25° C. to about 40° C., about 30° C. to about 35° C., about 30° C. to about 37° C., or about 30° C. to about 40° C. prior to administration to a subject.

In some embodiments, a composition (e.g., ACF) of the disclosure is acclimatized to a temperature of about −80° C. to about 0° C. prior to administration to a subject. In some embodiments, a composition (e.g., ACF) of the disclosure is acclimatized to a temperature of about −40° C. to about 0° C. prior to administration to a subject. In some embodiments, a composition (e.g., ACF) of the disclosure is acclimatized to a temperature of about −20° C. to about 0° C. prior to administration to a subject.

In some embodiments, a composition (e.g., ACF) of the disclosure is acclimatized to a temperature of about 2° C. to about 8° C. prior to administration to a subject. In some embodiments, a composition (e.g., ACF) of the disclosure is acclimatized to a temperature of about 1° C. to about 12° C. prior to administration to a subject. In some embodiments, a composition (e.g., ACF) of the disclosure is acclimatized to a temperature of about 4° C. to about 6° C. prior to administration to a subject.

In some embodiments, a composition (e.g., ACF) of the disclosure is acclimatized to a temperature of about 16° C. to about 30° C. prior to administration to a subject. In some embodiments, a composition (e.g., ACF) of the disclosure is acclimatized to a temperature of about 18° C. to about 25° C. prior to administration to a subject. In some embodiments, a composition (e.g., ACF) of the disclosure is acclimatized to a temperature of about 20° C. to about 25° C. prior to administration to a subject.

In some embodiments, a composition (e.g., ACF) of the disclosure is incubated (e.g., stored) at a first temperature, then acclimatized to a different temperature prior to administration to a subject.

In some embodiments, a composition (e.g., ACF) of the disclosure is incubated (e.g., stored) at a temperature of at least about −90° C., at least about −80° C., at least about −70° C., at least about −60° C., at least about −50° C., at least about −40° C., at least about −30° C., at least about −20° C., at least about −10° C., at least about −5° C., at least about −2° C., at least about −1° C., at least about 0° C., at least about 1° C., at least about 2° C., at least about 4° C., at least about 6° C., at least about 8° C., at least about 10° C., at least about 12° C., at least about 14° C., at least about 16° C., at least about 18° C., at least about 20° C., at least about 23° C., at least about 25° C., at least about 30° C., at least about 35° C., at least about 37° C., or at least about 40° C., then acclimatized to a different temperature disclosed herein prior to administration to a subject.

In some embodiments, a composition (e.g., ACF) of the disclosure is incubated (e.g., stored) at a temperature of at most about −90° C., at most about −80° C., at most about −70° C., at most about −60° C., at most about −50° C., at most about −40° C., at most about −30° C., at most about −20° C., at most about −10° C., at most about −5° C., at most about −2° C., at most about −1° C., at most about 0° C., at most about 1° C., at most about 2° C., at most about 4° C., at most about 6° C., at most about 8° C., at most about 10° C., at most about 12° C., at most about 14° C., at most about 16° C., at most about 18° C., at most about 20° C., at most about 23° C., at most about 25° C., at most about 30° C., at most about 35° C., at most about 37° C., or at most about 40° C., then acclimatized to a different temperature disclosed herein prior to administration to a subject.

In some embodiments, a composition (e.g., ACF) of the disclosure is incubated (e.g., stored) at a temperature of at most about −20° C., then acclimatized to a different temperature disclosed herein prior to administration to a subject. In some embodiments, a composition (e.g., ACF) of the disclosure is incubated (e.g., stored) at a temperature of at most about −10° C., then acclimatized to a different temperature disclosed herein prior to administration to a subject. In some embodiments, a composition (e.g., ACF) of the disclosure is incubated (e.g., stored) at a temperature of at most about 0° C., then acclimatized to a different temperature disclosed herein prior to administration to a subject. In some embodiments, a composition (e.g., ACF) of the disclosure is incubated (e.g., stored) at a temperature of at most about 2° C., then acclimatized to a different temperature disclosed herein prior to administration to a subject. In some embodiments, a composition (e.g., ACF) of the disclosure is incubated (e.g., stored) at a temperature of at most about 4° C., then acclimatized to a different temperature disclosed herein prior to administration to a subject. In some embodiments, a composition (e.g., ACF) of the disclosure is incubated (e.g., stored) at a temperature of at most about 8° C., then acclimatized to a different temperature disclosed herein prior to administration to a subject.

In some embodiments, a composition (e.g., ACF) of the disclosure is incubated (e.g., stored) at a temperature of about −90° C., about −80° C., about −70° C., about −60° C., about −50° C., about −40° C., about −30° C., about −20° C., about −10° C., about −5° C., about −2° C., about −1° C., about 0° C., about 1° C., about 2° C., about 4° C., about 6° C., about 8° C., about 10° C., about 12° C., about 14° C., about 16° C., about 18° C., about 20° C., about 23° C., about 25° C., about 30° C., about 35° C., about 37° C., or about 40° C., then acclimatized to a different temperature disclosed herein prior to administration to a subject.

In some embodiments, a composition (e.g., ACF) of the disclosure is incubated (e.g., stored) at a temperature of about −90° C. to about −80° C., about −90° C. to about −70° C., about −90° C. to about −60° C., about −90° C. to about −50° C., about −90° C. to about −40° C., about −90° C. to about −30° C., about −90° C. to about −20° C., about −90° C. to about −10° C., about −90° C. to about −5° C., about −90° C. to about −2° C., about −90° C. to about −1° C., about −90° C. to about 0° C., about −90° C. to about 1° C., about −90° C. to about 2° C., about −90° C. to about 4° C., about −90° C. to about 6° C., about −90° C. to about 8° C., about −90° C. to about 10° C., about −90° C. to about 12° C., about −90° C. to about 14° C., about −90° C. to about 16° C., about −90° C. to about 18° C., about −90° C. to about 20° C., about −90° C. to about 23° C., about −90° C. to about 25° C., about −90° C. to about 30° C., about −90° C. to about 35° C., about −90° C. to about 37° C., about −90° C. to about 40° C., about −40° C. to about −30° C., about −40° C. to about −20° C., about −40° C. to about −10° C., about −40° C. to about −5° C., about −40° C. to about −2° C., about −40° C. to about −1° C., about −40° C. to about 0° C., about −40° C. to about 1° C., about −40° C. to about 2° C., about −40° C. to about 4° C., about −40° C. to about 6° C., about −40° C. to about 8° C., about −40° C. to about 10° C., about −40° C. to about 12° C., about −40° C. to about 14° C., about −40° C. to about 16° C., about −40° C. to about 18° C., about −40° C. to about 20° C., about −40° C. to about 23° C., about −40° C. to about 25° C., about −40° C. to about 30° C., about −40° C. to about 35° C., about −40° C. to about 37° C., about −40° C. to about 40° C., about −20° C. to about −10° C., about −20° C. to about −5° C., about −20° C. to about −2° C., about −20° C. to about −1° C., about −20° C. to about 0° C., about −20° C. to about 1° C., about −20° C. to about 2° C., about −20° C. to about 4° C., about −20° C. to about 6° C., about −20° C. to about 8° C., about −20° C. to about 10° C., about −20° C. to about 12° C., about −20° C. to about 14° C., about −20° C. to about 16° C., about −20° C. to about 18° C., about −20° C. to about 20° C., about −20° C. to about 23° C., about −20° C. to about 25° C., about −20° C. to about 30° C., about −20° C. to about 35° C., about −20° C. to about 37° C., about −20° C. to about 40° C., about −10° C. to about −5° C., about −10° C. to about −2° C., about −10° C. to about −1° C., about −10° C. to about 0° C., about −10° C. to about 1° C., about −10° C. to about 2° C., about −10° C. to about 4° C., about −10° C. to about 6° C., about −10° C. to about 8° C., about −10° C. to about 10° C., about −10° C. to about 12° C., about −10° C. to about 14° C., about −10° C. to about 16° C., about −10° C. to about 18° C., about −10° C. to about 20° C., about −10° C. to about 23° C., about −10° C. to about 25° C., about −10° C. to about 30° C., about −10° C. to about 35° C., about −10° C. to about 37° C., about −10° C. to about 40° C., about 0° C. to about 0° C., about 0° C. to about 1° C., about 0° C. to about 2° C., about 0° C. to about 4° C., about 0° C. to about 6° C., about 0° C. to about 8° C., about 0° C. to about 10° C., about 0° C. to about 12° C., about 0° C. to about 14° C., about 0° C. to about 16° C., about 0° C. to about 18° C., about 0° C. to about 20° C., about 0° C. to about 23° C., about 0° C. to about 25° C., about 0° C. to about 30° C., about 0° C. to about 35° C., about 0° C. to about 37° C., about 0° C. to about 40° C., about 2° C. to about 4° C., about 2° C. to about 6° C., about 2° C. to about 8° C., about 2° C. to about 10° C., about 2° C. to about 12° C., about 2° C. to about 14° C., about 2° C. to about 16° C., about 2° C. to about 18° C., about 2° C. to about 20° C., about 2° C. to about 23° C., about 2° C. to about 25° C., about 2° C. to about 30° C., about 2° C. to about 35° C., about 2° C. to about 37° C., about 2° C. to about 40° C., about 4° C. to about 6° C., about 4° C. to about 8° C., about 4° C. to about 10° C., about 4° C. to about 12° C., about 4° C. to about 14° C., about 4° C. to about 16° C., about 4° C. to about 18° C., about 4° C. to about 20° C., about 4° C. to about 23° C., about 4° C. to about 25° C., about 4° C. to about 30° C., about 4° C. to about 35° C., about 4° C. to about 37° C., about 4° C. to about 40° C., about 8° C. to about 10° C., about 8° C. to about 12° C., about 8° C. to about 14° C., about 8° C. to about 16° C., about 8° C. to about 18° C., about 8° C. to about 20° C., about 8° C. to about 23° C., about 8° C. to about 25° C., about 8° C. to about 30° C., about 8° C. to about 35° C., about 8° C. to about 37° C., about 8° C. to about 40° C., about 12° C. to about 14° C., about 12° C. to about 16° C., about 12° C. to about 18° C., about 12° C. to about 20° C., about 12° C. to about 23° C., about 12° C. to about 25° C., about 12° C. to about 30° C., about 12° C. to about 35° C., about 12° C. to about 37° C., about 12° C. to about 40° C., about 20° C. to about 23° C., about 20° C. to about 25° C., about 20° C. to about 30° C., about 20° C. to about 35° C., about 20° C. to about 37° C., about 20° C. to about 40° C., about 23° C. to about 25° C., about 23° C. to about 30° C., about 23° C. to about 35° C., about 23° C. to about 37° C., about 23° C. to about 40° C., about 25° C. to about 30° C., about 25° C. to about 35° C., about 25° C. to about 37° C., about 25° C. to about 40° C., about 30° C. to about 35° C., about 30° C. to about 37° C., or about 30° C. to about 40° C., then acclimatized to a different temperature disclosed herein prior to administration to a subject.

In some embodiments, a composition (e.g., ACF) of the disclosure is incubated (e.g., stored) at a temperature of about −80° C. to about 0° C., then acclimatized to a different temperature disclosed herein prior to administration to a subject. In some embodiments, a composition (e.g., ACF) of the disclosure is incubated (e.g., stored) at a temperature of about −40° C. to about 0° C., then acclimatized to a different temperature disclosed herein prior to administration to a subject. In some embodiments, a composition (e.g., ACF) of the disclosure is incubated (e.g., stored) at a temperature of about −20° C. to about 0° C., then acclimatized to a different temperature disclosed herein prior to administration to a subject.

In some embodiments, a composition (e.g., ACF) of the disclosure is incubated (e.g., stored) at a temperature below 0° C. (e.g., about −80° C. to about 0° C., or about −40° C. to about 0° C.), then acclimatized to an ambient temperature or room temperature (e.g., about 15° C. to about 25° C., about 18° C. to about 25° C., or about 20° C. to about 25° C.) prior to administration to a subject.

In some embodiments, a composition (e.g., ACF) of the disclosure is incubated (e.g., stored) at a temperature of about 2° C. to about 8° C., then acclimatized to a different temperature disclosed herein prior to administration to a subject. In some embodiments, a composition (e.g., ACF) of the disclosure is incubated (e.g., stored) at a temperature of about 1° C. to about 12° C., then acclimatized to a different temperature disclosed herein prior to administration to a subject. In some embodiments, a composition (e.g., ACF) of the disclosure is incubated (e.g., stored) at a temperature of about 4° C. to about 6° C., then acclimatized to a different temperature disclosed herein prior to administration to a subject.

In some embodiments, a composition (e.g., ACF) of the disclosure is incubated (e.g., stored) at a temperature of about 2° C. to about 8° C., then acclimatized to an ambient temperature or room temperature (e.g., about 15° C. to about 25° C., about 18° C. to about 25° C., or about 20° C. to about 25° C.) prior to administration to a subject.

In some embodiments, a composition (e.g., ACF) of the disclosure is incubated (e.g., stored) at a temperature of about 16° C. to about 30° C., then acclimatized to a different temperature disclosed herein prior to administration to a subject. In some embodiments, a composition (e.g., ACF) of the disclosure is incubated (e.g., stored) at a temperature of about 18° C. to about 25° C., then acclimatized to a different temperature disclosed herein prior to administration to a subject. In some embodiments, a composition (e.g., ACF) of the disclosure is incubated (e.g., stored) at a temperature of about 20° C. to about 25° C., then acclimatized to a different temperature disclosed herein prior to administration to a subject.

Acclimatizing can comprise incubating the composition at the desired temperature for any suitable amount of time. In some embodiments, the composition is acclimatized for a time sufficient to bring the liquid formulation to a desired temperature, such as the temperature at which the composition is acclimatized. For example, in some embodiments the composition is acclimatized for a time sufficient to bring the liquid formulation to within about 1 degree, within about 2, within about 3 degrees, within about 4 degrees, within about 5 degrees, within about 6 degrees, within about 7 degrees, within about 8 degrees, within about 9 degrees, within about 10 degrees, within about 15 degrees, within about 20 degrees, or within about 25 degrees Celsius of the desired temperature, such as the temperature at which the composition is acclimatized. In some embodiments, acclimatizing the pharmaceutical composition comprises storing the pharmaceutical composition at room temperature for about 1 minute, about 3 minutes, about 5 minutes, about 10 minutes, about 15 minutes, about 20 minutes, about 30 minutes, or about 1 hour.

In some embodiments, acclimatizing comprises incubating the composition at the desired temperature for at least about 10 seconds, at least about 15 seconds, at least about 30 seconds, at least about 1 minute, at least about 3 minutes, at least about 5 minutes, at least about 10 minutes, at least about 15 minutes, at least about 20 minutes, at least about 30 minutes, or at least about 1 hour.

In some embodiments, acclimatizing comprises incubating the composition at the desired temperature for about 10 seconds, about 15 seconds, about 30 seconds, about 1 minute, about 3 minutes, about 5 minutes, about 10 minutes, about 15 minutes, about 20 minutes, about 30 minutes, or about 1 hour.

In some embodiments, acclimatizing comprises incubating the composition at a temperature disclosed herein that results in the formulation partially freezing or fully freezing, then allowing the formulation to thaw prior to administration, for example, by incubating the pharmaceutical composition at room temperature for about 1 minute, about 3 minutes, about 5 minutes, about 10 minutes, about 15 minutes, about 20 minutes, about 30 minutes, or about 1 hour prior to administration.

Suitable storage and limiting freeze-thaw cycles can allow maintained functionality of one or more bioactive agent(s) in a composition (e.g., ACF) disclosed herein. A composition (e.g., ACF) disclosed herein can be first subjected to long term storage frozen, then can upon a first use or after moving to a higher temperature, the composition can exhibit suitable stability for storage at a refrigerated temperature. For example, in some embodiments, a composition (e.g., ACF) of the disclosure is first incubated (e.g., stored) at a temperature below 0° C. (e.g., about −80° C. to about 0° C., or about −40° C. to about 0° C.), then after a first use or after moving to a higher temperature, the composition is subsequently incubated (e.g., stored) at a temperature of about 2° C. to about 8° C. for at most about one month. In some embodiments, a composition (e.g., ACF) of the disclosure is first incubated (e.g., stored) at a temperature below 0° C. (e.g., about −80° C. to about 0° C., or about −40° C. to about 0° C.), then after a first use or after moving to a higher temperature, the composition is subsequently incubated (e.g., stored) at a temperature of about 2° C. to about 8° C. for at most about two weeks. In some embodiments, a composition (e.g., ACF) of the disclosure is first incubated (e.g., stored) at a temperature below 0° C. (e.g., about −80° C. to about 0° C., or about −40° C. to about 0° C.), then after a first use or after moving to a higher temperature, the composition is subsequently incubated (e.g., stored) at a temperature of about 2° C. to about 8° C. for at most about one week. In some embodiments, a composition (e.g., ACF) of the disclosure is first incubated (e.g., stored) at a temperature below 0° C. (e.g., about −80° C. to about 0° C., or about −40° C. to about 0° C.), then after a first use or after moving to a higher temperature, the composition is subsequently incubated (e.g., stored) at a temperature of about 2° C. to about 8° C. for at most about three days.

### Clinical Evaluation

Various clinical evaluation techniques can be used to evaluate suitability of a subject for treatment with a composition (e.g., ACF) of the disclosure, and/or to monitor treatment efficacy or disease progression.

### Corneal Fluorescein Staining of Cornea

A degree of total corneal epithelial cell injury can be evaluated, for example, by corneal fluorescein staining. Fluorescein staining of the cornea can be performed by first placing a drop of sterile saline on a sterile fluorescein strip. The fluorescein can then be placed in the inferior cul de sac of the eye by pulling down on the lower lid and gently touching the bulbar conjunctiva with the fluorescein strip. The patient blinks to distribute the dye, and the cobalt blue light is used to determine whether any corneal epithelial defects exist. The water-soluble dye can enter and stain the corneal stroma in areas where the epithelium is absent or where intercellular junctions are loose. Such observations can be useful for detecting corneal injury. Each zone of the cornea (central, superior, inferior, medial, and lateral) can be assessed, for example, according to criteria in TABLE 3. In some embodiments, one or more of the zones is excluded from the scoring.

TABLE 3

| Score | Description |
|---|---|
| 0 | No punctate stain in the area |
| 0.5 | 1-5 micropuntate stain spots |
| 1.0 | 6-15 micropunctate stain spots |
| 1.5 | More than 15 micropuntate stain spots |
| 2.0 | Moderate macropunctate stain spots (involving less than 50% of |
| | the area) |
| 2.5 | Moderate macropunctate stain spots (involving more than 50% of |
| | the area) |
| 3.0 | Clumped macropunctate stain spots (involving less than 50% of |
| | the cornea) |
| 3.5 | Clumped macropunctate stain spots (involving more than 50% of |
| | the cornea) |
| 4.0 | Severe diffuse (coalescent) macropuntate stain of the area |

In some embodiments, a subject has a fluorescein corneal staining sum score of at least 1, least 2, at least 3, at least 4, at least 5, at least 6, at least 7, at least 8, at least 9, at least 10, at least 11, at least 12, at least 13, at least 14, at least 15, at least 16, at least 17, or at least 18 for one eye prior to treatment with a composition of the disclosure. In some embodiments, a subject's fluorescein corneal staining sum score for one eye improves by at least 1, least 2, at least 3, at least 4, at least 5, at least 6, at least 7, at least 8, at least 9, at least 10, at least 11, at least 12, at least 13, at least 14, or at least 15 points following administration of a composition as disclosed herein.

In some embodiments, a subject has a combined fluorescein corneal staining sum score for both eyes of at least 4, at least 6, at least 8, at least 10, at least 12, at least 14, at least 16, at least 18, at least 20, at least 22, at least 24, at least 26, at least 28, or at least 30 prior to treatment with a composition of the disclosure. In some embodiments, a subject's combined fluorescein corneal staining sum score for both eyes improves by at least 1, at least 2, at least 3, at least 4, at least 5, at least 6, at least 7, at least 8, at least 9, at least 10, at least 11, at least 12, at least 13, at least 14, at least 15, at least 16, at least 18, at least 20, at least 22, at least 24, at least 26, or at least 28 points following administration of a composition as disclosed herein.

In some embodiments, a subject has a fluorescein corneal staining score for a single zone in one eye of at least 0.5, at least 1, at least 1.5, a least 2, at least 2.5, or at least 3 prior to treatment with a composition of the disclosure. In some embodiments, a subject's fluorescein corneal staining score for a single zone in one eye improves by at least 0.5, at least 1, at least 1.5, a least 2, at least 2.5, or at least 3 following administration of a composition as disclosed herein.

### Lissamine Green Conjunctival Staining

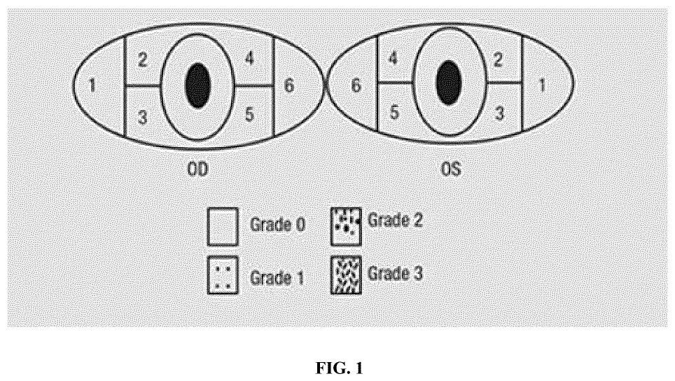

A degree of conjunctival injury can be evaluated, for example, by Lissamine green conjunctival staining. Lissamine green staining can highlight conjunctival epithelial cells that are damaged or dead. Lissamine green can be instilled, for example, at a concentration of 1% with a drop of >25 μl. A slit lamp biomicroscope can be used to observe this staining either with a white light, producing a blue-green stain, or with the use of an enhancing red barrier filter that produces a black appearance to the pattern of staining. Grading can be performed independently in each of the areas in FIG. 1 according to the National Eye Institute (NEI) scale (grade 0-3). In some embodiments, superior zones 2 and 4 are excluded from analysis.

In some embodiments, prior to treatment with a composition of the disclosure, a subject has a Lissamine green conjunctival staining sum score for one eye of at least 1, at least 2, at least 3, at least 4, at least 5, at least 6, at least 7, at least 8, at least 9, at least 10, or at least 11 out of a total possible score of 12 when superior zones 2 and 4 are excluded from analysis. In some embodiments, prior to treatment with a composition of the disclosure, a subject has a Lissamine green conjunctival staining sum score for one eye of ≥3 to ≤9 out of a total possible score of 12 when superior zones 2 and 4 are excluded from analysis. In some embodiments, the subject's Lissamine green conjunctival staining sum score for the one eye (excluding superior zones 2 and 4) improves by at least 1, least 2, at least 3, at least 4, at least 5, at least 6, at least 7, at least 8, at least 9, or at least 10 following administration of a composition as disclosed herein.

In some embodiments, prior to treatment with a composition of the disclosure, a subject has a Lissamine green conjunctival staining sum score for one eye of at least 1, least 2, at least 3, at least 4, at least 5, at least 6, at least 7, at least 8, at least 9, at least 10, at least 11, at least 12, at least 13, at least 14, at least 15, or at least 16 out of a total possible score of 18 when all zones are included. In some embodiments, the subject's Lissamine green conjunctival staining sum score for the one eye (including superior zones 2 and 4) improves by at least 1, least 2, at least 3, at least 4, at least 5, at least 6, at least 7, at least 8, at least 9, at least 10, at least 11, at least 12, at least 13, at least 14, at least 15, or at least 16 following administration of a composition as disclosed herein.

In some embodiments, prior to treatment with a composition of the disclosure, a subject has a Lissamine green conjunctival staining score for a single zone in one eye of at least 1, at least 2, or at least 3 prior to treatment with a composition of the disclosure. In some embodiments, the subject's Lissamine green conjunctival staining score for the single zone improves by at least 1, at least 2, or at least 3 following administration of a composition as disclosed herein.

### Visual Analogue Scale

A visual analogue scale can be used to evaluate the amount of eye dryness and/or irritation a subject is experiencing. For example, subjects can be asked to indicate the amount of eye dryness and/or irritation they are experiencing on a scale from 0 (no dryness/irritation) to 100 (severe dryness/irritation).

In some embodiments, prior to treatment with a composition of the disclosure, a subject reports a visual analog scale score of at least 20, at least 30, at least 40, at least 50, at least 60, at least 70, at least 80, or at least 90 on a scale from 0 (no dryness/irritation) to 100 (severe dryness/irritation). In some embodiments, the subject's visual analogue scale score is improved by at least 5, at least 10, at least 15, at least 20, at least 25, at least 30, at least 40, at least 50, at least 60, at least 70, at least 80 following administration of a composition as disclosed herein.

### Ocular Surface Disease Index (OSDI) Score

An Ocular Surface Disease Index (OSDI) score can be used to evaluate a subject's symptoms of an ocular surface disease. The Ocular Surface Disease Index (OSDI) is a 12-item questionnaire designed to provide a rapid assessment of the symptoms of ocular irritation consistent with dry eye disease and their impact on vision-related functioning. An OSDI questionnaire can comprise the following questions, with answers graded at a 0-4 scale (0=none of the time; 1=some of the time, 2-half of the time; 3=most of the time; 4-all of the time):

A. Have you experienced any of the following during the last week? (1) eyes that are sensitive to light; (2) Eyes that feel gritty; (3) painful or sore eye; (4) blurred vision; (5) poor vision.

B. Have problems with your eyes limited you in performing any of the following during the last week? (6) reading; (7) driving at night; (8) working with a computer or bank ATM; (9) watching TV.

C. Have your eyes felt uncomfortable in any of the following situation in the last week? (10) windy conditions; (11) places or areas with low humidity (very dry); (12) areas that are air conditioned.

The scores are tallied to generate a total Ocular Surface Disease Index score.

In some embodiments, a subject has an OSDI score of at least 10, at least 15, at least 20, at least 25, at least 30, at least 25, or at least 40 prior to treatment with a composition of the disclosure.

In some embodiments, administering a pharmaceutical formulation (e.g., ACF formulation) as disclosed herein can result in an average improvement in an OSDI score of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 25, or at least 30 points.

### Visual Acuity (VA)

Visual acuity (VA) is acuteness or clearness of vision that is dependent on the sharpness of the retinal focus within the eye and the sensitivity of the interpretative faculty of the brain. Visual acuity is a measure of the spatial resolution of the visual processing system. VA can be tested by requiring the subject whose vision is being tested to identify characters, typically numbers or letters, on a chart from a set distance. Chart characters are represented as black symbols against a white background. The distance between the person's eyes and the testing chart is set at a sufficient distance to approximate infinity in the way the lens attempts to focus. Twenty feet, or six meters, can be essentially infinity from an optical perspective. In the present disclosure, an improvement in visual acuity can be assessed by an increase in the number of letters read from the chart.

In some embodiments, the disclosure provides a method for increasing visual acuity, the method comprising administering to a subject in need thereof a formulation disclosed herein. In some embodiments, the increase in visual acuity can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 letters. The increase in visual acuity can be about 1 letter, about 5 letters, about 10 letters, about 15 letters, about 20 letters, or about 25 letters. In some embodiments, the disclosure provides a method for increasing the number of letters recognizable by a treated eye from about 1 to about 30 letters. In some embodiments, the number of letters recognizable is increased from about 5 to about 25 letters, from about 5 to about 20 letters, from about 5 to about 15 letters, from about 5 to about 10 letters, from about 10 to about 25 letters, from about 15 to about 25 letters, from about 20 to about 25 letters.

One non-limiting example of a test for measuring Visual Acuity is the use of the ESV-3000 ETDRS testing device self-calibrated test lighting. The ESV-3000 device incorporates LED light source technology. The auto-calibration circuitry constantly monitors the LED light source and calibrates the test luminance to 85 cd/m² or 3 cd/m². To evaluate ETDRS properly, the test should be conducted under standardized lighting conditions, for, example, photopic test level of 85 cd/m². After providing a baseline evaluation, the increase or decrease in the number of letters that can be identified by the test subject provides a measure of sight increase or decrease during treatment.

In some embodiments, improvement of clinical symptoms is monitored, for example, by indirect ophthalmoscopy, fundus photography, fluorescein angiography, electroretinography, external eye examination, slit lamp biomicroscopy, applanation tonometry, pachymetry, optical coherence tomography, autorefraction, or a combination thereof.

### OPAS Score

In some embodiments, an Ocular Pain Assessment Survey (OPAS) is used to evaluate a subject or an effect of a composition of the disclosure. OPAS is a validated questionnaire for assessing ocular pain severity, non-ocular pain severity, quality of life (QOL), impact of aggravating factors (e.g., mechanical stimuli, such as wind, dry air, heat, and air conditioning, and chemical stimuli, such as volatile chemicals, fumes, and cosmetic fragrance), and associated factors.

### Combination Therapies

A pharmaceutical composition provided herein can be administered in conjunction with other therapeutic agents, for example, other ophthalmic preparations. In some embodiments, a pharmaceutical composition disclosed herein is administered in combination with a prescription dry eye therapeutic agent. In some embodiments, a pharmaceutical composition disclosed herein is administered in combination with on over the counter dry eye therapeutic agent.

The other therapeutic agents can be administered prior to, after, or concomitantly with the pharmaceutical composition.

Multiple therapeutic agents disclosed herein can be administered in any order or simultaneously. If simultaneously, the multiple therapeutic agents can be provided in a single, unified form, or in multiple forms, for example, as multiple separate eye drops, or as eye drops and injectables. The therapeutic agents can be packed together or separately, in a single package or in a plurality of packages. One or all of the therapeutic agents can be given in multiple doses. If not simultaneous, the timing between the multiple doses can vary, for example, to as much as about a month.

In some embodiments, a pharmaceutical composition provided herein is administered as a monotherapy, e.g., as the only therapeutic agent to treat a particular condition in a subject in need thereof.

### Embodiments

Embodiment 1. A pharmaceutical formulation comprising in a mixture: (a) about 1-10 mg/mL of amniotic membrane; (b) about 10-40% v/v amniotic fluid; and (c) a pharmaceutically-acceptable excipient.

Embodiment 2. The pharmaceutical formulation of embodiment 1, wherein the pharmaceutical formulation comprises about 5 mg/mL of the amniotic membrane.

Embodiment 3. The pharmaceutical formulation of embodiment 1 or embodiment 2, wherein the pharmaceutical formulation comprises about 20 to about 30% v/v of the amniotic fluid.

Embodiment 4. The pharmaceutical formulation of embodiment 1 or embodiment 2, wherein the pharmaceutical formulation comprises about 20% v/v of the amniotic fluid.

Embodiment 5. The pharmaceutical formulation of embodiment 1 or embodiment 2, wherein the pharmaceutical formulation comprises about 25% v/v of the amniotic fluid.

Embodiment 6. The pharmaceutical formulation of embodiment 1 or embodiment 2, wherein the pharmaceutical formulation comprises about 30% v/v of the amniotic fluid.

Embodiment 7. The pharmaceutical formulation of any one of embodiments 1-6, wherein the pharmaceutically-acceptable excipient is a balanced salt solution (BSS).

Embodiment 8. The pharmaceutical formulation of any one of embodiments 1-7, wherein the amniotic fluid is human amniotic fluid from about a full term pregnancy.

Embodiment 9. The pharmaceutical formulation of any one of embodiments 1-7, wherein the amniotic fluid is human amniotic fluid from a third trimester of pregnancy.

Embodiment 10. The pharmaceutical formulation of any one of embodiments 1-9, wherein the pharmaceutical formulation has a density of about 1 g/mL to about 1.01 g/mL.

Embodiment 11. The pharmaceutical formulation of any one of embodiments 1-10, wherein the pharmaceutical formulation has a density of about 1.005 g/mL to about 1.006 g/mL.

Embodiment 12. The pharmaceutical formulation of any one of embodiments 1-11, wherein the pharmaceutical formulation is contained in a bottle component of an eye dropper and the pharmaceutical formulation is formulated to be dispensed from the eye dropper.

Embodiment 13. The pharmaceutical formulation of embodiment 12, wherein the eye dropper is a multi-dose eye dropper.

Embodiment 14. The pharmaceutical formulation of embodiment 12 or embodiment 13, wherein the eye dropper contains at least about 1 mL of the pharmaceutical formulation.

Embodiment 15. The pharmaceutical formulation of any one of embodiments 12-14, wherein the eye dropper contains about 1 mL to about 15 mL of the pharmaceutical formulation.

Embodiment 16. The pharmaceutical formulation of any one of embodiments 12-14, wherein the eye dropper contains about 10 mL of the pharmaceutical formulation.

Embodiment 17. The pharmaceutical formulation of any one of embodiments 12-14, wherein the eye dropper contains about 3 mL of the pharmaceutical formulation.

Embodiment 18. The pharmaceutical formulation of any one of embodiments 12-17, wherein the eye dropper further comprises a nozzle component that comprises a one-way valve that reduces back-flow of the pharmaceutical formulation into the eye dropper upon dispensing a dose.

Embodiment 19. The pharmaceutical formulation of embodiment 18, wherein the nozzle component further comprises a semi-permeable membrane that facilities intake of sterile air into the eye dropper upon dispensing the dose.

Embodiment 20. The pharmaceutical formulation of any one of embodiments 12-19, wherein the pharmaceutical formulation does not contain a preservative.

Embodiment 21. The pharmaceutical formulation of any one of embodiments 12-20, wherein the eye dropper delivers a dose of about 15 microliters to about 60 microliters.

Embodiment 22. The pharmaceutical formulation of any one of embodiments 12-20, wherein the eye dropper delivers a dose of about 20 microliters to about 40 microliters.

Embodiment 23. The pharmaceutical formulation of any one of embodiments 12-20, wherein the eye dropper delivers a dose of about 30 microliters.

Embodiment 24. The pharmaceutical formulation of any one of embodiments 12-23, wherein the eye dropper delivers a dose upon application of an actuation force of less than 35 N.

Embodiment 25. The pharmaceutical formulation of any one of embodiments 12-23, wherein the eye dropper delivers a dose upon application of an actuation force of about 10 N to about 30 N.

Embodiment 26. The pharmaceutical formulation of any one of embodiments 12-25, wherein the one-way valve comprises an outlet orifice with a diameter of about 1 to about 4 mm.

Embodiment 27. The pharmaceutical formulation of any one of embodiments 12-26, wherein the one-way valve comprises an outlet orifice with a diameter of about 1.5 to about 2.5 mm.

Embodiment 28. The pharmaceutical formulation of any one of embodiments 12-27, wherein the nozzle component further comprises an antimicrobial agent that reduces likelihood of microbial contamination.

Embodiment 29. The pharmaceutical formulation of embodiment 28, wherein the eye dropper further comprises a cap component that comprises the antimicrobial agent that reduces likelihood of microbial contamination.

Embodiment 30. The pharmaceutical formulation of any one of embodiments 12-29, wherein the bottle component comprises low-density polyethylene (LDPE).

Embodiment 31. A method of treating a condition in a subject in need thereof, the method comprising: (a) acclimatizing a pharmaceutical formulation at a temperature of at most about 30° C., wherein the pharmaceutical formulation comprises amniotic fluid and a pharmaceutically-acceptable excipient; and (b) administering the pharmaceutical formulation to the subject.

Embodiment 32. The method of embodiment 31, wherein the acclimatizing is at a temperature of at most about 0° C.

Embodiment 33. The method of embodiment 31 or embodiment 32, wherein the acclimatizing comprises incubating the pharmaceutical formulation at the temperature for a time sufficient for the pharmaceutical formulation to reach a temperature of at most about 0° C.

Embodiment 34. The method of embodiment 31, wherein the acclimatizing is at a temperature of about 1° C. to about 10° C.

Embodiment 35. The method of embodiment 34, wherein the acclimatizing comprises incubating the pharmaceutical formulation at the temperature for a time sufficient for the pharmaceutical formulation to reach a temperature of about 1° C. to about 10° C.

Embodiment 36. The method of embodiment 31, wherein the acclimatizing is at a temperature of about 20° C. to about 25° C.

Embodiment 37. The method of embodiment 36, wherein the acclimatizing comprises incubating the pharmaceutical formulation at the temperature for a time sufficient for the pharmaceutical formulation to reach a temperature of about 20° C. to about 25° C.

Embodiment 38. The method of any one of embodiments 31-37, wherein the acclimatizing comprises incubating the pharmaceutical formulation at the temperature for at least about 1 minute.

Embodiment 39. The method of any one of embodiments 31-38, wherein the pharmaceutical formulation further comprises amniotic membrane.

Embodiment 40. The method of embodiment 39, wherein the pharmaceutical formulation comprises at least about 0.1 mg/mL of the amniotic membrane.

Embodiment 41. The method of any one of embodiments 31-40, wherein the pharmaceutical formulation comprises at least about 1% v/v of the amniotic fluid.

Embodiment 42. A method of treating a condition in a subject in need thereof, the method comprising administering to the subject the pharmaceutical formulation of any one of embodiments 1-30.

Embodiment 43. The method of embodiment 42, wherein the condition is mild dry eye syndrome.

Embodiment 44. The method of embodiment 42, wherein the condition is moderate dry eye syndrome.

Embodiment 45. The method of embodiment 42, wherein the condition is severe dry eye syndrome.

Embodiment 46. The method of any one of embodiments 42-45, wherein the pharmaceutical formulation is administered at least once every three days.

Embodiment 47. The method of any one of embodiments 42-46, wherein the pharmaceutical formulation is administered at least once per day.

Embodiment 48. The method of any one of embodiments 42-47, wherein the pharmaceutical formulation is administered two times per day.

Embodiment 49. The method of any one of embodiments 42-48, wherein the pharmaceutical formulation is administered topically.

Embodiment 50. The method of any one of embodiments 42-49, wherein the pharmaceutical formulation is administered in doses of about 15 microliters to about 60 microliters.

Embodiment 51. The method of any one of embodiments 42-49, wherein the pharmaceutical formulation is administered in doses of about 20 microliters to about 40 microliters.

Embodiment 52. The method of any one of embodiments 42-49, wherein the pharmaceutical formulation is administered in doses of about 30 microliters.

Embodiment 53. The method of any one of embodiments 42-52, wherein after 30 days of treatment, the subject exhibits a reduction in a degree of total corneal epithelial cell injury for at least one zone of one eye as determined by corneal fluorescein staining with scoring according to criteria in Table 3.

Embodiment 54. The method of any one of embodiments 42-53, wherein after 30 days of treatment, the subject exhibits a reduction in conjunctival injury for at least one zone of one eye as determined by Lissamine green conjunctival staining with scoring according to a National Eye Institute scale.

Embodiment 55. The method of any one of embodiments 42-54, wherein the subject exhibits an improvement in a visual analogue score after 30 days of treatment.

Embodiment 56. The method of any one of embodiments 42-55, wherein the subject exhibits an improvement in total Ocular Surface Disease Index score after 30 days of treatment.

Embodiment 57. The method of any one of embodiments 42-56, wherein a visual acuity of the subject improves by at least 5 letters after 30 days of treatment.

Embodiment 58. An eye dropper device comprising: (a) a bottle component containing a pharmaceutical formulation that comprises amniotic fluid and a pharmaceutically-acceptable excipient in a mixture; and (b) a nozzle component attached to the bottle component and configured to dispense a dose of the pharmaceutical formulation from the bottle component, wherein the nozzle component comprises a one-way valve that obstructs back-flow of liquid into the bottle component upon dispensing the dose.

Embodiment 59. The eye dropper device of embodiment 58, wherein the pharmaceutical formulation further comprises amniotic membrane.

Embodiment 60. The eye dropper device of embodiment 58 or embodiment 59, wherein the nozzle component further comprises a semi-permeable membrane that facilities intake of sterile air into the eye dropper upon dispensing the dose.

Embodiment 61. The eye dropper device of any one of embodiments 58-60, wherein the pharmaceutical formulation does not contain a preservative.

Embodiment 62. The eye dropper device of any one of embodiments 58-61, wherein the one-way valve comprises an outlet orifice with a diameter of about 1 mm to about 4 mm.

Embodiment 63. The eye dropper device of any one of embodiments 58-61, wherein the one-way valve comprises an outlet orifice with a diameter of about 1.5 mm to about 2.5 mm.

Embodiment 64. The eye dropper device of any one of embodiments 58-63, wherein the nozzle component further comprises an antimicrobial agent that reduces likelihood of microbial contamination.

Embodiment 65. The eye dropper device of embodiment 64, further comprising a cap component that comprises the antimicrobial agent that reduces likelihood of microbial contamination, wherein the cap component covers the nozzle component and is configured to be removed and replaced.

Embodiment 66. The eye dropper device of any one of embodiments 58-65, wherein the bottle component comprises low-density polyethylene (LDPE).

Embodiment 67. The eye dropper device of any one of embodiments 58-66, wherein the dose is about 15 microliters to about 60 microliters.

Embodiment 68. The eye dropper device of any one of embodiments 58-66, wherein the dose is about 20 microliters to about 40 microliters.

Embodiment 69. The eye dropper device of any one of embodiments 58-66, wherein the dose is about 30 microliters.

Embodiment 70. The eye dropper device of any one of embodiments 58-69, wherein the eye dropper delivers the dose upon application of an actuation force to the bottle component of less than 35 N.

Embodiment 71. The eye dropper device of any one of embodiments 58-69, wherein the eye dropper delivers the dose upon application of an actuation force to the bottle component of about 10 N to about 30 N.

Embodiment 72. The eye dropper device of any one of embodiments 58-71, wherein the pharmaceutical formulation further comprises at least about 0.1 mg/mL of amniotic membrane.

Embodiment 73. The eye dropper device of any one of embodiments 58-72, wherein the pharmaceutical formulation comprises at least about 1% v/v of the amniotic fluid.

Embodiment 74. The eye dropper device of any one of embodiments 58-73, wherein the eye dropper device contains at least 1 mL of the pharmaceutical formulation.

Embodiment 75. The eye dropper device of any one of embodiments 58-74, wherein the eye dropper device contains about 1 to about 15 mL of the pharmaceutical formulation.

Embodiment 76. The eye dropper device of any one of embodiments 58-75, wherein the eye dropper device contains about 10 mL of the pharmaceutical formulation.

Embodiment 77. The eye dropper device of any one of embodiments 58-75, wherein the eye dropper device contains about 3 mL of the pharmaceutical formulation.

Embodiment 78. The eye dropper device of any one of embodiments 58-77, wherein the pharmaceutical formulation is the pharmaceutical formulation of any one of embodiments 1-30.

Embodiment 79. A pharmaceutical formulation comprising at least 5 bioactive agents in table 2, each at a concentration of at least 0.1 pg/mL, and a pharmaceutically-acceptable excipient in a mixture.

In some embodiments, the invention provides a pharmaceutical formulation comprising in a mixture: (a) about 1-10 mg/mL of amniotic membrane; (b) about 10-40% v/v amniotic fluid; and (c) a pharmaceutically-acceptable excipient. In some embodiments, the pharmaceutical formulation comprises about 5 mg/mL of the amniotic membrane. In some embodiments, the pharmaceutical formulation comprises about 20 to about 30% v/v of the amniotic fluid. In some embodiments, the pharmaceutical formulation comprises about 20% v/v of the amniotic fluid. In some embodiments, the pharmaceutical formulation comprises about 25% v/v of the amniotic fluid. In some embodiments, the pharmaceutical formulation comprises about 30% v/v of the amniotic fluid. In some embodiments, the pharmaceutically-acceptable excipient is a balanced salt solution (BSS). In some embodiments, the amniotic fluid is human amniotic fluid from a second trimester of pregnancy. In some embodiments, the amniotic fluid is human amniotic fluid from a third trimester of pregnancy. In some embodiments, the pharmaceutical formulation has a density of about 1 g/mL to about 1.01 g/mL. In some embodiments, the pharmaceutical formulation has a density of about 1.005 g/mL to about 1.006 g/mL. In some embodiments, the pharmaceutical formulation is contained in a bottle component of an eye dropper and the pharmaceutical formulation is formulated to be dispensed from the eye dropper. In some embodiments, the eye dropper is a multi-dose eye dropper. In some embodiments, the eye dropper contains at least about 1 mL of the pharmaceutical formulation. In some embodiments, the eye dropper contains about 1 mL to about 15 mL of the pharmaceutical formulation. In some embodiments, the eye dropper contains about 10 mL of the pharmaceutical formulation. In some embodiments, the eye dropper contains about 3 mL of the pharmaceutical formulation. In some embodiments, the eye dropper further comprises a nozzle component that comprises a one-way valve that reduces a back-flow of the pharmaceutical formulation into the eye dropper upon dispensing a dose. In some embodiments, the nozzle component further comprises a semi-permeable membrane that facilities intake of sterile air into the eye dropper upon dispensing a dose. In some embodiments, the pharmaceutical formulation does not contain a preservative. In some embodiments, the eye dropper delivers a dose of about 15 microliters to about 60 microliters. In some embodiments, the eye dropper delivers a dose of about 20 microliters to about 40 microliters. In some embodiments, the eye dropper delivers a dose of about 30 microliters. In some embodiments, the eye dropper delivers a dose upon application of an actuation force of less than 35 N. In some embodiments, the eye dropper delivers a dose upon application of an actuation force of about 10 N to about 30 N. In some embodiments, the one-way valve comprises an outlet orifice with a diameter of about 1 to about 4 mm. In some embodiments, the one-way valve comprises an outlet orifice with a diameter of about 1.5 to about 2.5 mm. In some embodiments, the nozzle component further comprises an antimicrobial agent that reduces likelihood of microbial contamination. In some embodiments, the eye dropper further comprises a cap component that comprises an antimicrobial agent that reduces likelihood of microbial contamination. In some embodiments, the bottle component comprises low-density polyethylene (LDPE).

In some embodiments, the invention provides a method of treating a condition in a subject in need thereof, the method comprising: (a) acclimatizing a pharmaceutical formulation at a temperature of at most about 30° C., wherein the pharmaceutical formulation comprises amniotic fluid and a pharmaceutically-acceptable excipient; and (b) administering the pharmaceutical formulation to the subject. In some embodiments, the acclimatizing is at a temperature of about at most about 0° C. In some embodiments, the acclimatizing comprises incubating the pharmaceutical formulation at the temperature for a time sufficient for the pharmaceutical formulation to reach a temperature of at most about 0° C. In some embodiments, the acclimatizing is at a temperature of about 1° C. to about 10° C. In some embodiments, the acclimatizing comprises incubating the pharmaceutical formulation at the temperature for a time sufficient for the pharmaceutical formulation to reach a temperature of about 1° C. to about 10° C. In some embodiments, the acclimatizing is at a temperature of about 20° C. to about 25° C. In some embodiments, the acclimatizing comprises incubating the pharmaceutical formulation at the temperature for a time sufficient for the pharmaceutical formulation to reach a temperature of about 20° C. to about 25° C. In some embodiments, the acclimatizing comprises incubating the pharmaceutical formulation at the temperature for at least about 1 minute. In some embodiments, the pharmaceutical formulation further comprises amniotic membrane. In some embodiments, the pharmaceutical formulation comprises at least about 0.1 mg/mL of the amniotic membrane. In some embodiments, the pharmaceutical formulation comprises at least about 1% v/v of the amniotic fluid.

In some embodiments, the invention provides a method of treating a condition in a subject in need thereof, the method comprising administering to the subject the pharmaceutical formulation as disclosed herein. In some embodiments, the condition is mild dry eye syndrome. In some embodiments, the condition is moderate dry eye syndrome. In some embodiments, the condition is severe dry eye syndrome. In some embodiments, the pharmaceutical formulation is administered at least once every three days. In some embodiments, the pharmaceutical formulation is administered at least once per day. In some embodiments, the pharmaceutical formulation is administered two times per day. In some embodiments, the pharmaceutical formulation is administered topically. In some embodiments, the pharmaceutical formulation is administered in doses of about 15 microliters to about 60 microliters. In some embodiments, the pharmaceutical formulation is administered in doses of about 20 microliters to about 40 microliters. In some embodiments, the pharmaceutical formulation is administered in doses of about 30 microliters. In some embodiments, after 30 days of treatment, the subject exhibits a reduction in a degree of total corneal epithelial cell injury for at least one zone of one eye as determined by corneal fluorescein staining with scoring according to criteria in Table 3. In some embodiments, wherein after 30 days of treatment, the subject exhibits a reduction in conjunctival injury for at least one zone of one eye as determined by Lissamine green conjunctival staining with scoring according to a National Eye Institute scale. In some embodiments, the subject exhibits an improvement in a visual analog score after 30 days of treatment. In some embodiments, the subject exhibits an improvement in total Ocular Surface Disease Index score after 30 days of treatment. In some embodiments, a visual acuity of the subject improves by at least 5 letters after 30 days of treatment. In some embodiments, the pharmaceutical formulation is the pharmaceutical formulation as described herein.

In some embodiments, the invention provides an eye dropper device comprising: (a) a bottle component containing a pharmaceutical formulation that comprises amniotic fluid and a pharmaceutically-acceptable excipient in a mixture; and (b) a nozzle component attached to the bottle component and configured to dispense a dose of the pharmaceutical formulation from the bottle component, wherein the nozzle component comprises a one-way valve that obstructs back-flow of liquid into the bottle component upon dispensing the dose. In some embodiments, the pharmaceutical formulation further comprises amniotic membrane. In some embodiments, the nozzle component further comprises a semi-permeable membrane that facilities intake of sterile air into the eye dropper upon dispensing the dose. In some embodiments, the pharmaceutical formulation does not contain a preservative. In some embodiments, the one-way valve comprises an outlet orifice with a diameter of about 1 mm to about 4 mm. In some embodiments, the one-way valve comprises an outlet orifice with a diameter of about 1.5 mm to about 2.5 mm. In some embodiments, the nozzle component further comprises an antimicrobial agent that reduces likelihood of microbial contamination. In some embodiments, the eye dropper device further comprises a cap component that comprises an antimicrobial agent that reduces likelihood of microbial contamination, wherein the cap component covers the nozzle component and is configured to be removed and replaced. In some embodiments, the bottle component comprises low-density polyethylene (LDPE). In some embodiments, the dose is about 15 microliters to about 60 microliters. In some embodiments, the dose is about 20 microliters to about 40 microliters. In some embodiments, the dose is about 30 microliters. In some embodiments, the eye dropper delivers the dose upon application of an actuation force to the bottle component of less than 35 N. In some embodiments, the eye dropper delivers the dose upon application of an actuation force to the bottle component of about 10 N to about 30 N. In some embodiments, the pharmaceutical formulation comprises at least about 0.1 mg/mL of amniotic membrane. In some embodiments, the pharmaceutical formulation comprises at least about 1% v/v of amniotic fluid. In some embodiments, the eye dropper device contains at least 1 mL of the pharmaceutical formulation. In some embodiments, the eye dropper device contains about 1 to about 15 mL of the pharmaceutical formulation. In some embodiments, the eye dropper device contains about 10 mL of the pharmaceutical formulation. In some embodiments, the eye dropper device contains about 3 mL of the pharmaceutical formulation.

In some embodiments, the invention provides a pharmaceutical formulation comprising at least 5 bioactive agents in table 2, each at a concentration of at least 0.1 pg/mL, and a pharmaceutically-acceptable excipient in a mixture.

### Examples

### EXAMPLE 1: Amniotic cytokine formulation (ACF) Formulations

Amniotic fluid was collected as part of elective cesarean section procedures. Amniotic membrane was collected from placental tissue obtained during elective cesarean section procedures. Amniotic fluid and homogenized amniotic membrane were combined with balanced salt solution (BSS) to generate Amniotic cytokine formulations (ACF) as indicated in TABLE 4. The formulations have densities of approximately 1.005-1.006 g/mL.

TABLE 4

| | ACF | | ACF | | ACF | |
|---|---|---|---|---|---|---|
| | Formulation | | Formulation | | Formulation | |
| | R | | A | | G | |
| Amnion membrane | 0.375 | g | 0.375 | g | 0.375 | g |
| Amniotic fluid | 15 | mL | 18.75 | mL | 22.5 | mL |
| BSS | 60 | mL | 56.25 | mL | 52. | mL |

### EXAMPLE 2: Characterization of Amniotic cytokine formulation (ACF) Formulations

The ACFs generated in EXAMPLE 1 were diluted four-fold and the concentrations of various bioactive agents were measured by array-based multiplex enzyme-linked immunosorbent assay (ELISA), with two replicates for ACF formulation R and ACF formulation A, and three replicates for ACF formulation G. TABLE 5 provides the measured concentrations in pg/mL.

TABLE 5: concentrations of proteins in Amniotic cytokine formulation (ACF) formulations after four-fold dilution. Data are in pg/mL. LOD: limit of detection. MAX: maximum value. AR: Amphiregulin; BDNF: Brain-derived neurotrophic factor; bFGF: Fibroblast growth factor 2; BLC: C—X—C motif chemokine 13/B lymphocyte chemoattractant; BMP-4: Bone morphogenetic protein 4; BMP-5: Bone morphogenetic protein 5; BMP-7: Bone morphogenetic protein 7; β-NGF: Beta-nerve growth factor; EGF: Pro-epidermal growth factor [Cleaved into: Epidermal growth factor (Urogastrone)]; EGF R: Epidermal growth factor receptor; EG-VEGF: Prokineticin-1/Endocrine-gland-derived vascular endothelial growth factor; FGF-4: Fibroblast growth factor 4; FGF-7: Fibroblast growth factor 7; G-CSF: Granulocyte colony-stimulating factor; GDF-15: Growth/differentiation factor 15/Macrophage inhibitory cytokine 1 (MIC-1); GDNF: Glial cell line-derived neurotrophic factor; GH: Somatotropin/Growth hormone; GM-CSF: Granulocyte-macrophage colony-stimulating factor; HB-EGF: Proheparin-binding EGF-like growth factor [Cleaved into: Heparin-binding EGF-like growth factor; HGF: Hepatocyte growth factor; I-309: C-C motif chemokine 1/T lymphocyte-secreted protein I-309; ICAM-1: Intercellular adhesion molecule 1; IFN-γ: Interferon gamma; IGF-1: Insulin-like growth factor I; IGFBP-1: Insulin-like growth factor-binding protein 1; IGFBP-2: Insulin-like growth factor-binding protein 2; IGFBP-3: Insulin-like growth factor-binding protein 3; IGFBP-4: Insulin-like growth factor-binding protein 4; IGFBP-6: Insulin-like growth factor-binding protein 6; IL-10: Interleukin-10; IL-11: Interleukin-11; IL-12p40: Interleukin-12 subunit beta; IL-12p70: Interleukin-12 subunit alpha; IL-13: Interleukin-13; IL-15: Interleukin-15; IL-16: Interleukin-16; IL-17: Interleukin-17A; IL-1a: Interleukin-1 alpha; IL-1β: Interleukin-1 beta; IL-1ra: Interleukin-1 receptor antagonist protein; IL-2: Interleukin-2; IL-4: Interleukin-4; IL-5: Interleukin-5; IL-6: Interleukin-6; IL-6R: Interleukin-6 receptor subunit alpha; IL-7: Interleukin-7; IL-8: Interleukin-8; MCP-1: C-C motif chemokine 2/Monocyte chemoattractant protein 1; MCSF: Macrophage colony-stimulating factor 1; MCSF R: Macrophage colony-stimulating factor 1 receptor; MIG: C—X—C motif chemokine 9/Monokine induced by interferon-gamma; MIP-1α: C-C motif chemokine 3/Macrophage inflammatory protein 1-alpha; MIP-1β: C-C motif chemokine 4/Macrophage inflammatory protein 1-beta; MIP-1δ: C-C motif chemokine 15/Macrophage inflammatory protein 5; NGF R: Tumor necrosis factor receptor superfamily member 16/Low-affinity nerve growth factor receptor; NT-3: Neurotrophin-3; NT-4: Neurotrophin-4; OPG: Tumor necrosis factor receptor superfamily member 11B/;Osteoprotegerin; PDGF-AA: Platelet-derived growth factor subunit A; PDGF-BB: Platelet-derived growth factor subunit B; PIGF: Placenta growth factor; RANTES: C-C motif chemokine 5; SCF: Kit ligand/Stem cell factor; SCF R: Mast/stem cell growth factor receptor Kit; TGFα: Protransforming growth factor alpha [Cleaved into: Transforming growth factor alpha]; TGFβ1: Transforming growth factor beta-1; TGFβ3: Transforming growth factor beta-3; TIMP-1: Metalloproteinase inhibitor 1/Tissue inhibitor of metalloproteinases 1; TIMP-2: Metalloproteinase inhibitor 2/Tissue inhibitor of metalloproteinases 2; TNF RI: Tumor necrosis factor receptor superfamily member 1A/Tumor necrosis factor receptor 1; TNF RII: Tumor necrosis factor receptor superfamily member 1B/Tumor necrosis factor receptor 2; TNFα: Tumor necrosis factor/Tumor necrosis factor ligand superfamily member 2; TNFβ: Lymphotoxin-alpha/Tumor necrosis factor ligand superfamily member 1; VEGF: Vascular endothelial growth factor A; VEGF R2: Vascular endothelial growth factor receptor 2; VEGF R3: Vascular endothelial growth factor receptor 3; VEGF-D: Vascular endothelial growth factor D.

TABLE 5

| | | | ACF Formulation | | | | | | |
|---|---|---|---|---|---|---|---|---|---|
| Analyte | LOD | MAX | R | R | A | A | G | G | G |
| AR | 4.3 | 1,111.1 | 8.3 | 7.8 | 11.6 | 11.6 | 17.3 | 7.5 | 15.8 |
| BDNF | 0.3 | 666.7 | 0 | 0 | 0.1 | 0.4 | 0.3 | 0 | 0 |
| bFGF | 0.8 | 740.7 | 0 | 0 | 0.8 | 1 | 3.8 | 0 | 0.9 |
| BLC | 0.2 | 222.2 | 2.7 | 3 | 1.1 | 0.8 | 2.1 | 4 | 2.4 |
| BMP-4 | 42 | 11,111.1 | 0 | 0 | 0 | 0 | 70.9 | 0 | 0 |
| BMP-5 | 1,650.3 | 100,000 | 0 | 0 | 0 | 0 | 707 | 0 | 0 |
| BMP-7 | 9.9 | 40,000 | 0 | 1.4 | 5.1 | 5.1 | 13.4 | 0 | 4.7 |
| β-NGF | 1 | 3,333.3 | 8 | 8.4 | 0 | 0.5 | 2.5 | 0 | 13.4 |
| EGF | 0 | 66.7 | 2.7 | 2.9 | 2.5 | 2.9 | 2.9 | 4.1 | 3 |
| EGF R | 1.4 | 3,333.3 | 82.1 | 79.6 | 186.7 | 180.6 | 235.5 | 78.6 | 130.1 |
| EG- | 0.9 | 3,333.3 | 9.3 | 9.9 | 12.6 | 12.8 | 47 | 17.9 | 12.5 |
| VEGF | | | | | | | | | |
| Eotaxin | 0.2 | 148.1 | 0.4 | 0 | 0.2 | 0.2 | 0.4 | 0.5 | 0 |
| Eotaxin- | 1.5 | 1,000 | 2.6 | 1.4 | 5.3 | 3.6 | 4 | 2.6 | 0.7 |
| 2 | | | | | | | | | |
| FGF-4 | 36.9 | 11,111.1 | 83.4 | 93.8 | 14.9 | 0 | 66 | 0 | 34.9 |
| FGF-7 | 12.8 | 3,333.3 | 0 | 0 | 2.1 | 9.6 | 147.8 | 0 | 0 |
| G-CSF | 1.6 | 2,222.2 | 13.9 | 9.4 | 7.6 | 10.7 | 14.9 | 7.8 | 8.7 |
| GDF-15 | 0.5 | 666.7 | 472.5 | 590.1 | 541.7 | 489.5 | 826.1 | 513.3 | 436.9 |
| GDNF | 6.2 | 4,000 | 0 | 0 | 0 | 0 | 11 | 0 | 0 |
| GH | 3.3 | 370.4 | 0 | 0 | 0.3 | 0 | 4.6 | 0 | 0 |
| GM- | 1.7 | 1,000 | 1.4 | 1.7 | 1.6 | 3 | 0.5 | 1.3 | 1.7 |
| CSF | | | | | | | | | |
| HB- | 0.9 | 370.4 | 1.4 | 0.3 | 1.1 | 1.8 | 6.7 | 0 | 3.4 |
| EGF | | | | | | | | | |
| HGF | 7.2 | 4,000 | 397 | 387.4 | 268.1 | 294.6 | 1,854.6 | 219.5 | 262.2 |
| I-309 | 1.2 | 1,333.3 | 1.3 | 0 | 1.2 | 0.3 | 3.5 | 1.2 | 0.1 |
| ICAM-1 | 5.7 | 11,111.1 | 5,427.3 | 5,206.9 | 4,398.5 | 4,289.7 | 6,122.5 | 5,144.2 | 4,499.4 |
| IFN-Υ | 2.5 | 2,000 | 0 | 0 | 0 | 0.2 | 0.2 | 0.9 | 0 |
| IGF-1 | 8.1 | 740.7 | 0 | 0 | 0 | 0 | 0 | 0 | 0 |
| IGFBP- | 4.4 | 1,666.7 | 2,619.4 | 2,777 | 2,909.5 | 2,709.3 | 4,298.3 | 2,749.7 | 3,258.4 |
| 1 | | | | | | | | | |
| IGFBP- | 5.8 | 6,666.7 | 4,953.7 | 4,729.1 | 5,356.5 | 4,180.3 | 4,219.7 | 4,581.1 | 3,916.9 |
| 2 | | | | | | | | | |
| IGFBP- | 129.3 | 66,666.7 | 39,531.5 | 41,691.7 | 33,798.8 | 36,891.8 | 53,580.8 | 32,039.5 | 39,819 |
| 3 | | | | | | | | | |
| IGFBP- | 232.9 | 66,666.7 | 38,630.6 | 44,698.7 | 71,992.1 | 62,525.3 | 93,991.1 | 61,817.1 | 86,272.6 |
| 4 | | | | | | | | | |
| IGFBP- | 12.1 | 3,703.7 | 3,729.5 | 3,572.8 | 4,522.5 | 3,953.7 | 5,278.5 | 3,773.5 | 4,835.2 |
| 6 | | | | | | | | | |
| IL-10 | 0.3 | 1,333.3 | 0.5 | 0.2 | 0.5 | 0 | 0.1 | 0.3 | 0.4 |
| IL-11 | 1.9 | 740.7 | 0.8 | 0 | 0 | 0.5 | 0.5 | 0 | 0 |
| IL- | 0.2 | 3,333.3 | 0.9 | 0.6 | 0.7 | 0.3 | 1.6 | 0.9 | 0.9 |
| 12p40 | | | | | | | | | |
| IL- | 0.1 | 500 | 0.1 | 0 | 0 | 0.1 | 0 | 0.1 | 0 |
| 12p70 | | | | | | | | | |
| IL-13 | 0.2 | 333.3 | 0.2 | 0 | 0 | 0.2 | 0.1 | 0.1 | 0.1 |
| IL-15 | 0.6 | 4,000 | 0 | 0.2 | 0.2 | 0.4 | 0.7 | 0.3 | 0 |
| IL-16 | 0.7 | 5,000 | 1.2 | 0.7 | 1.2 | 1.5 | 0.5 | 0.8 | 1.1 |
| IL-17 | 1.2 | 1,333.3 | 0.4 | 0.3 | 1 | 0.6 | 0.3 | 0.3 | 0.7 |
| IL-1α | 2.6 | 666.7 | 3.6 | 3.3 | 7.8 | 5.2 | 5.4 | 8.6 | 4.8 |
| IL-1β | 0.4 | 333.3 | 1.3 | 1.3 | 1.9 | 1.4 | 1 | 1.2 | 1.3 |
| IL-1rα | 0.9 | 222.2 | 44.8 | 53.5 | 62.6 | 58.2 | 55.5 | 50.9 | 38 |
| IL-2 | 5 | 2,000 | 0.2 | 0 | 0 | 2.1 | 3.1 | 3.3 | 1.9 |
| IL-4 | 3 | 666.7 | 4.6 | 0 | 7.7 | 7.1 | 0 | 8.6 | 1.5 |
| IL-5 | 2 | 1,333.3 | 0 | 0.6 | 0 | 1.2 | 2.3 | 0 | 0 |
| IL-6 | 1.8 | 2,000 | 7.5 | 6.2 | 9.4 | 9.2 | 13.1 | 6.7 | 15.7 |
| IL-6R | 6.8 | 3,333.3 | 70.4 | 54.8 | 149.8 | 113.5 | 180.3 | 66.7 | 126.9 |
| IL-7 | 0.6 | 1,333.3 | 0.2 | 0.2 | 0 | 0.6 | 0.2 | 0.2 | 0.5 |
| IL-8 | 0.5 | 166.7 | 4.4 | 3.9 | 13.7 | 10.9 | 5 | 3.5 | 14.8 |
| Insulin | 3.3 | 2,222.2 | 0 | 0 | 1.2 | 0 | 17.7 | 0 | 0 |
| MCP-1 | 6.4 | 2,000 | 46.9 | 60.7 | 88.3 | 92.2 | 92.4 | 52.6 | 139.2 |
| MCSF | 0.5 | 1,333.3 | 112.4 | 116.7 | 88.5 | 100.7 | 95 | 56.8 | 99.7 |
| MCSF | 10.3 | 13,333.3 | 566.8 | 565.4 | 413 | 417.6 | 1,694.9 | 359.6 | 903.8 |
| R | | | | | | | | | |
| MIG | 1.3 | 1,666.7 | 3.6 | 3.3 | 1.6 | 6.6 | 6.1 | 3.5 | 38.6 |
| MIP-1α | 0.3 | 3,333.3 | 2.8 | 2.2 | 1.9 | 2.2 | 0.5 | 1.6 | 2.9 |
| MIP-1β | 0.2 | 333.3 | 2.1 | 1.8 | 2.2 | 2.8 | 1.7 | 1.1 | 3.2 |
| MIP-1δ | 0.2 | 1,111.1 | 89.7 | 86.7 | 42.2 | 63.5 | 8.6 | 108.5 | 78.9 |
| NGF R | 4.9 | 3,333.3 | 29.5 | 28.4 | 17.1 | 16.6 | 56.5 | 21.9 | 22.3 |
| NT-3 | 11.4 | 13,333.3 | 6.4 | 0 | 7.5 | 12.6 | 24.4 | 0 | 10.1 |
| NT-4 | 6.2 | 3,333.3 | 12.8 | 16.5 | 23.4 | 22.8 | 31.1 | 0.2 | 2.3 |
| OPG | 6 | 4,000 | 32.6 | 22.2 | 0 | 0 | 46.2 | 0 | 8.3 |
| PDGF- | 0.8 | 370.4 | 11.4 | 12.1 | 10.2 | 12.5 | 26 | 8.7 | 7.5 |
| AA | | | | | | | | | |
| PDGF- | 0.3 | 666.7 | 4.3 | 3.4 | 13.5 | 20.6 | 2.1 | 8.2 | 2.2 |
| BB | | | | | | | | | |
| PIGF | 1 | 444.4 | 0 | 0 | 0.1 | 0 | 3.6 | 0 | 0 |
| RANTES | 0.4 | 2,222.2 | 18.1 | 16.6 | 42.4 | 56.4 | 16.2 | 17.3 | 34.7 |
| SCF | 4 | 3,333.3 | 24.6 | 22.7 | 10.1 | 10 | 12.4 | 2.3 | 13.7 |
| SCF R | 4.7 | 6,666.7 | 25.3 | 25.4 | 18.7 | 13.8 | 60 | 12.2 | 37.1 |
| TGFα | 0.2 | 370.4 | 0 | 0 | 0 | 0 | 0.2 | 0 | 0 |
| TGFβ1 | 2,757.9 | 100,000 | 0 | 0 | 1,405 | 0 | 6,536 | 638.4 | 402 |
| TGFβ3 | 13.8 | 13,333.3 | 0 | 0 | 0 | 0 | 37.8 | 0 | 0 |
| TIMP-1 | 0.8 | 1,481.5 | 1,657.9 | 1,727.2 | 1,848.1 | 1,835.3 | 2,091 | 1,792.9 | 1,692.9 |
| TIMP-2 | 3.9 | 4,444.4 | 6,250.1 | 7,077 | 7,214.4 | 6,510.7 | 8,781.1 | 6,132.2 | 6,758.9 |
| TNF RI | 4.6 | 4,444.4 | 804.5 | 790.7 | 1,519.2 | 1,525.7 | 909.1 | 738.4 | 987.4 |
| TNF | 3.1 | 4,444.4 | 896.3 | 1,025.1 | 1,170.8 | 1,358.1 | 1,271.2 | 982.6 | 1,129.5 |
| RII | | | | | | | | | |
| TNFα | 4.8 | 666.7 | 1.8 | 0 | 0 | 0 | 0 | 2.3 | 0 |
| TNFβ | 21.6 | 20,000 | 0 | 2.7 | 8 | 15.9 | 21.4 | 30.1 | 0 |
| VEGF | 2.5 | 3,333.3 | 1.8 | 0 | 0.4 | 3.2 | 15.9 | 0.8 | 0 |
| VEGF | 27.1 | 10,000 | 9.9 | 0 | 54.5 | 61.3 | 135 | 0 | 18.8 |
| R2 | | | | | | | | | |
| VEGF | 4.2 | 13,333.3 | 3.3 | 0 | 0.6 | 0 | 17.8 | 16.1 | 0 |
| R3 | | | | | | | | | |
| VEGF- | 1.8 | 6,666.7 | 0.9 | 0.5 | 0.5 | 4 | 4 | 0 | 1.7 |
| D | | | | | | | | | |

### EXAMPLE 3: Evaluation of ACF Formulations in multi-dose eye droppers

The ACF formulations from EXAMPLE 1 were tested in multi-dose eye droppers. The eye droppers were calibrated to allow the formulations to be stored in a sterile multi-dose form without any preservatives to reduce toxicity and potential negative effects on the bioactive components of the ACF formulations. The eyedroppers contain a one-way valve that dispenses a dose and prevents back-flow of the solution into the bottle. The nozzle contains a semi-permeable silicone membrane to allow sterile air intake after a drop is delivered, without the need for standard filtration. The nozzle is colored to improve precision of drop delivery to the eye. The cap and nozzle top contain silver ions to reduce the likelihood of microbial contamination and growth on the external parts that come into contact with the liquid formulation. The bottle housing is made from low density polyethylene (LDPE), while the nozzle and cap are made from high density polyethylene (HDPE) and silicone rubber.

ACF formulation R was tested in 11 mL bottles filled with 10 mL of formulation R. ACF formulation A was tested in 11 ml bottles filled with 10 mL of formulation A. ACF formulation G was tested in 5 mL bottles filled with 3 mL of formulation G.

Drop size tests were conducted using nozzle valves with outlet orifices of various diameters to determine the average, minimum, and maximum size drops delivered. The sizes of 10 drops were calculated for each setup. The tests were conducted at 22.4-22.8° C., 59-65% humidity, and a pressure of 1.01×105 Pa. TABLE 6 provides the results.

TABLE 6
results of drop size test.

| | | Valve outlet orifice diameter | | | | | |
|---|---|---|---|---|---|---|---|
| Form. | | 1.6 mm | 2 mm | 2.4 mm | 2.7 mm | 3 mm | 3.6 mm |
| R | Average | 30.24 | 35.39 | 41.61 | 43.72 | 47.3 | 54.97 |
| | drop size | | | | | | |
| | SD | 0.42 | 0.61 | 0.62 | 0.37 | 0.62 | 0.33 |
| | Minimum | 29.62 | 34.59 | 40.76 | 43.14 | 46.42 | 54.37 |
| | drop size | | | | | | |
| | Maximum | 31.01 | 36.38 | 42.54 | 44.33 | 48.11 | 55.37 |
| | drop size | | | | | | |
| A | Average | 30.03 | 35.23 | 41.01 | 43.18 | 47.39 | 54.34 |
| | drop size | | | | | | |
| | SD | 0.37 | 0.57 | 0.35 | 0.41 | 0.47 | 0.4 |
| | Minimum | 29.45 | 34.33 | 40.5 | 42.49 | 46.67 | 53.43 |
| | drop size | | | | | | |
| | Maximum | 30.65 | 36.32 | 41.69 | 43.68 | 48.06 | 54.83 |
| | drop size | | | | | | |
| G | Average | 28.43 | 32.68 | 39.21 | 42.18 | 47.14 | 53.82 |
| | drop size | | | | | | |
| | SD | 0.38 | 0.6 | 0.51 | 0.28 | 0.63 | 0.46 |
| | Minimum | 27.73 | 31.71 | 38.17 | 41.85 | 46.02 | 52.98 |
| | drop size | | | | | | |
| | Maximum | 28.93 | 33.5 | 39.86 | 42.64 | 48.01 | 54.47 |
| | drop size | | | | | | |

Form. = ACF Formulation. SD = standard deviation. All values are in microliters.

Flow control tests were conducted to determine the risk of squeezing the eyedropper leading to a jet of liquid rather than a drop. Nozzle configurations designed for controlled delivery of very low viscosity (1-10 cP), low viscosity (10-200 cP), and high viscosity (200-1500 cP) liquids were tested. The nozzle configurations each had a 2.4 mm outlet orifice. An actuation force of 35N was used at actuation speeds from 50-250 mm/min. Jet formation upon manual actuation was also tested. The actuation force required to elicit a drop was also tested.

For ACF formulation R tested in an 11 mL bottle: (i) the 1-10 cP nozzle configuration removed the risk of jet but the actuation force could be close to 35N by the end of use of the device; (ii) the 10-200 cP nozzle configuration did not completely remove the risk of jet for a patient who can strongly and quickly actuate the device but the actuation force was acceptable; and (iii) the 200-1500 cP nozzle configuration did not remove the risk of jet but the actuation force was acceptable.

For ACF formulation R tested in a softer (thinner-walled) 11 mL bottle: (i) the 1-10 cP nozzle configuration did not completely remove the risk of jet for a patient who can strongly and quickly actuate the device but the actuation force was acceptable; (ii) the 10-200 cP nozzle configuration did not remove the risk of jet but the actuation force was acceptable; and (iii) the 200-1500 cP nozzle configuration did not remove the risk of jet but the actuation force was acceptable.

For ACF formulation A tested in an 11 mL bottle: (i) the 1-10 cP nozzle configuration removed the risk of jet but the actuation force could be close to 35N by the end of use of the device; (ii) the 10-200 cP nozzle configuration did not remove the risk of jet but the actuation force was acceptable; and (iii) the 200-1500 cP nozzle configuration did not remove the risk of jet but the actuation force was acceptable.

For ACF formulation A tested in a softer (thinner-walled) 11 ml bottle: (i) the 1-10 cP nozzle configuration removed the risk of jet and the actuation force was acceptable; (ii) the 10-200 cP nozzle configuration did not completely remove the risk of jet for a patient who can strongly and quickly actuate the device but the actuation force was acceptable; and (iii) the 200-1500 cP nozzle configuration did not remove the risk of jet but the actuation force was acceptable.

For ACF formulation G tested in a 5 mL bottle: (i) the 1-10 cP nozzle configuration removed the risk of jet but the actuation force could be close to 35N by the end of use of the device; (ii) the 10-200 cP nozzle configuration did not completely remove the risk of jet for a patient who can strongly and quickly actuate the device but the actuation force was acceptable; and (iii) the 200-1500 cP nozzle configuration did not remove the risk of jet but the actuation force was acceptable.

TABLE 7
provides the results of the actuation force test for the
indicated formulations and bottle sizes configured with 1.6 mm diameter
valve outlet orifices and the 1-10 cP nozzle configuration.

| | ACF formulation | | ACF formulation | | | |
|---|---|---|---|---|---|---|
| | R, 11 mL softer | | A, 11 mL softer | | ACF formulation | |
| | bottle | | bottle | | G, 5 mL bottle | |
| | First | Last | First | Last | First | Last |
| | drop | drop | drop | drop | drop | drop |
| Average | 12 | 22 | 12.54 | 22.54 | 27.71 | 35.21 |
| actuation | | | | | | |
| force (N) | | | | | | |
| SD | 0.84 | 0.84 | 1.4 | 1.4 | 1.61 | 1.61 |

Use tests were conducted to simulate 3× daily use at 2 drops per use of the eyedroppers over two weeks, and to test for changes in drop size or actuation force over time. Eyedroppers were used with valves comprising 1.6 mm diameter outlet orifices and the 1-10 cP nozzle configuration. TABLE 8 shows changes in average drop size and actuation force after two weeks of use.

TABLE 8

| | ACF formulation | | ACF formulation | | | |
|---|---|---|---|---|---|---|
| | R, 11 mL softer | | A, 11 mL softer | | ACF formulation | |
| | bottle | | bottle | | G, 5 mL bottle | |
| | Day 0 | Day 14 | Day 0 | Day 14 | Day 0 | Day 14 |
| Average | 30.24 | 36.87 | 30.03 | 35.8 | 28.43 | 31.61 |
| drop size | | | | | | |
| Drop size | 0.42 | 0.52 | 0.37 | 0.75 | 0.38 | 0.43 |
| SD | | | | | | |
| Average | 12 | 16.19 | 12.54 | 17.24 | 27.71 | 30.29 |
| actuation | | | | | | |
| force (N) | | | | | | |
| Actuation | 0.84 | 2.54 | 1.4 | 2.28 | 1.61 | 4 |
| force SD | | | | | | |

In the use test, a drop remnant was observed to stick to the nozzle of the eyedropper, potentially resulting in a false perception of device leak after several days of use. Additional use tests were conducted to assess whether shaking off the remaining drop remnant after each use and before closing the cap could improve performance. TABLE 9 shows changes in average drop size and actuation force after two weeks of use with the addition of a shaking action after use and prior to cap closure. In some cases, the shaking motion removed the remaining drop.

TABLE 9

| | ACF formulation | | ACF formulation | | | |
|---|---|---|---|---|---|---|
| | R, 11 mL softer | | A, 11 mL softer | | ACF formulation | |
| | bottle | | bottle | | G, 5 mL bottle | |
| | Day 0 | Day 14 | Day 0 | Day 14 | Day 0 | Day 14 |
| Average | 30.24 | 34.14 | 30.03 | 38.16 | 28.43 | 28.85 |
| drop size | | | | | | |
| Drop size | 0.42 | 0.52 | 0.37 | 0.85 | 0.38 | 0.35 |
| SD | | | | | | |
| Average | 11.82 | 14.54 | 11.74 | 14.68 | 28.17 | 30.62 |
| actuation | | | | | | |
| force (N) | | | | | | |
| Actuation | 0.43 | 2.49 | 1.47 | 1.81 | 0.88 | 3.17 |
| force SD | | | | | | |

Additional use tests were conducted to assess the effects of a vented cap on performance. TABLE 10 shows changes in average drop size and actuation force after two weeks of use with a vented cap.

TABLE 10

| | ACF formulation | | ACF formulation | | | |
|---|---|---|---|---|---|---|
| | R, 11 mL softer | | A, 11 mL softer | | ACF formulation | |
| | bottle | | bottle | | G, 5 mL bottle | |
| | Day 0 | Day 14 | Day 0 | Day 14 | Day 0 | Day 14 |
| Average | 30.24 | 33.67 | 30.03 | 32.94 | 28.43 | 33.03 |
| drop size | | | | | | |
| Drop size | 0.42 | 0.43 | 0.37 | 0.39 | 0.38 | 0.48 |
| SD | | | | | | |
| Average | 16.18 | 19.17 | 17.18 | 20.43 | 23.73 | 26.13 |
| actuation | | | | | | |
| force (N) | | | | | | |
| Actuation | 1.34 | 2.62 | 1.6 | 1.89 | 1.13 | 1.66 |
| force SD | | | | | | |

### EXAMPLE 4: Clinical study

A clinical study is performed to evaluate the safety and efficacy of the ACF formulations from EXAMPLE 1 for the treatment of mild, moderate, and severe Dry Eye Syndrome (DES).

Inclusion criteria for the study include the following: (i) subjects age 18 years or older on the date of informed consent; (ii) all subjects must provide signed written consent prior to participation in any study-related procedures; (iii) patient-reported history of keratoconjunctivitis sicca (KCS)/Dry Eye Syndrome (DES) for a period of at least 6 months; (iv) clinical diagnosis of bilateral KCS/DES supported by protocol-defined study assessments; (v) fluorescein corneal staining sum score of ≥3 to ≤9 (e.g., out of a total possible score of 12, or an equivalent score out of a total possible score of 20 for one eye or 40 for two eyes as disclosed herein), must have at least a score of 2 in one of the five zones in the same eye at Baseline; (vi) Lissamine green conjunctival staining sum score of ≥3 to ≤9 out of a total possible score of 12 (scoring excludes superior zones 2 and 4) in the same eye at Baseline; (vii) Ocular Surface Disease Index (OSDI) score ≥40 at Baseline; (viii) Snellen Visual Acuity (VA) of better than 20/200 in each eye; (ix) Willing to discontinue use of current dry eye therapy (including artificial tears or ocular lubricants) throughout the duration of the study (30 days); (x) Female subjects of childbearing potential must have a negative urine pregnancy test at Baseline.

Subjects are administered the formulations topically in both eyes, according to the dosing schedule in TABLE 11.

TABLE 11

| | | Dosing | Duration of |
|---|---|---|---|
| Treatment cohort | Size | frequency | treatment |
| ACF formulation R | 10 subjects | 2× per day (B.I.D.) | 30 days |
| ACF formulation A | 10 subjects | 2× per day (B.I.D.) | 30 days |
| ACF formulation G | 10 subjects | 2× per day (B.I.D.) | 30 days |

Subjects are evaluated at day 0, day 14, and day 30. The study eye is the eye with the higher fluorescein cornea staining score at baseline. If both eyes score equally, the right eye is chosen as the study eye. Both eyes are treated evenly if only one eye meets the eligibility criteria. Assessment criteria are summarized in TABLE 12.

TABLE 12
TABLE 12: study schedule.

| | Visit 1 | Visit 2 | Visit 3 |
|---|---|---|---|
| Assessment | Baseline | Day 14 + 2 | Day 30 + 2 |
| Visual Acuity (SNELLEN) | OU | OU | OU |
| Ocular Surface Disease Index | X | X | X |
| (OSDI) | | | |
| Corneal Fluorescein Staining | OU | OU | OU |
| Lissamine Green Dye Staining | OU | OU | OU |
| of Conjunctiva | | | |
| Visual Analog Scale for Eye | OU | OU | OU |
| Dryness | | | |
| Assess AE's | | X | X |
| Drug accountability | | | X |

OU = Both eyes, X = Perform study procedures.

### Ocular Surface Disease Index (OSDI)

Subjects answer the following questions, with answers graded at a 0-4 scale (0=none of the time; 1=some of the time, 2=half of the time; 3=most of the time; 4=all of the time):

A. Have you experienced any of the following during the last week? (1) eyes that are sensitive to light; (2) Eyes that feel gritty; (3) painful or sore eye; (4) blurred vision; (5) poor vision.

B. Have problems with your eyes limited you in performing any of the following during the last week? (6) reading; (7) driving at night; (8) working with a computer or bank ATM; (9) watching TV.

C. Have your eyes felt uncomfortable in any of the following situation in the last week? (10) windy conditions; (11) places or areas with low humidity (very dry); (12) areas that are air conditioned.

The scores are tallied to generate a total Ocular Surface Disease Index score.

### Corneal Fluorescein Staining

Corneal staining is performed to grade the degree of corneal epithelial cell injury as measured by fluorescence using slit-lamp examination. Fluorescein is instilled into the eye, and each zone of the cornea (central, superior, inferior, medial, and lateral) is assessed and scored at the slit lamp within 2.5-5 minutes of instillation, according to the criteria in TABLE 3. Both eyes are assessed.

### Lissamine Green Dye Staining of Conjunctiva

Lissamine Green is instilled approximately 5 minutes after corneal staining. Conjunctiva are evaluated approximately 1-4 minutes after Lissamine Green instillation. Low to moderate intensity of white light of the slit lamp is used. The conjunctiva to be evaluated is within the interpalpebral region of the nasal and temporal zones, and grading is to be performed independently in each of the areas in FIG. 1 according to the National Eye Institute (NEI) scale (grade 0-3). Once grading of the right eye is complete, the entire procedure is repeated for the left eye.

### Visual Analogue Sore

Subjects are asked to indicate the amount of eye dryness and/or irritation they are experiencing on a scale from 0 (no dryness/irritation) to 100 (severe dryness/irritation).

### Endpoints

Efficacy endpoints for the study include: (i) mean change from baseline to day 30 in total corneal fluorescein staining in the designated study eye; (ii) mean change from baseline to day 30 in visual analog Score and/or OPAS Survey score; (iii) mean change from baseline to day 30 in Lissamine green conjunctival staining in the study eye; and (iv) mean change from baseline to day 30 in OSDI (Ocular Surface Disease Index) score.

Safety endpoints for the study include visual acuity, and documented adverse events.

What is claimed is:

1. A method of treating an ocular surface disease in a subject in need thereof, the method comprising administering to the subject a pharmaceutical formulation comprising in a mixture:
(a) about 0.01-10 mg/mL of amniotic membrane;
(b) about 10-40% v/v amniotic fluid; and
(c) a pharmaceutically-acceptable excipient;
wherein the pharmaceutical formulation is contained in a bottle component of an eye dropper and the pharmaceutical formulation is formulated to be dispensed from the eye dropper;
wherein the eye dropper is configured to deliver a dose of about 20-50 microliters of the pharmaceutical formulation upon application of an actuation force of about 10-35 N.

2. The method of claim 1, wherein the eye dropper is configured to deliver a dose of about 31-39 microliters of the pharmaceutical formulation upon application of an actuation force of about 10-25 N.

3. The method of claim 1, wherein the eye dropper comprises a nozzle component, wherein the nozzle component comprises a channel configured to modulate a flow rate of the pharmaceutical formulation and reduce risk of jet formation upon applying the actuation force to the eyedropper.

4. The method of claim 3, wherein the nozzle component comprises a semi-permeable membrane, wherein the semi-permeable membrane facilities intake of air into the eye dropper upon dispensing a dose, wherein the air is sterile after passing into the eye dropper.

5. The method of claim 4, wherein the nozzle component further comprises a one-way valve that reduces back-flow of the pharmaceutical formulation into the eye dropper upon dispensing the dose.

6. The method of claim 5, wherein the one-way valve comprises an outlet orifice with a diameter of about 1.5 mm to about 2.5 mm.

7. The method of claim 1, wherein the method further comprises, prior to the administering: (i) storing the pharmaceutical formulation in the eye dropper at a temperature below −10° C., and (ii) acclimatizing the pharmaceutical formulation in the eye dropper to a temperature of about 16-37° C.

8. The method of claim 7, wherein the method further comprises storing the pharmaceutical formulation in the eye dropper at a temperature of 2-8° C.

9. The method of claim 8, wherein the pharmaceutical formulation in the eye dropper is stored at the temperature of 2-8° C. for at most two weeks.

10. The method of claim 1, wherein the pharmaceutical formulation comprises about 0.1-0.5 mg/mL of the amniotic membrane.

11. The method of claim 1, wherein the pharmaceutical formulation comprises about 25-35% v/v of the amniotic fluid.

12. The method of claim 1, wherein the pharmaceutical formulation comprises about 30% v/v of the amniotic fluid.

13. The method of claim 1, wherein the amniotic fluid is human amniotic fluid from a full term pregnancy.

14. The method of claim 1, wherein the pharmaceutically-acceptable excipient is a balanced salt solution (BSS).

15. The method of claim 1, wherein the pharmaceutical formulation has a density of about 1 g/mL to about 1.01 g/mL.

16. The method of claim 1, wherein the pharmaceutical formulation does not contain a preservative.

17. The method of claim 1, wherein the pharmaceutical formulation is administered 2-4 times per day.

18. The method of claim 1, wherein the ocular surface disease is dry eye syndrome.

19. A pharmaceutical formulation comprising in a mixture:
(a) about 0.01-10 mg/mL of amniotic membrane;
(b) about 10-40% v/v amniotic fluid; and
(c) a pharmaceutically-acceptable excipient.

20. A method of treating a condition in a subject in need thereof, the method comprising:
(a) storing a pharmaceutical formulation at a temperature below −10° C., wherein the pharmaceutical formulation comprises amniotic fluid and a pharmaceutically-acceptable excipient;
(b) acclimatizing the pharmaceutical formulation to a temperature of about 16-37° C.;
(c) administering the pharmaceutical formulation to the subject; and
(d) storing the pharmaceutical formulation at a temperature of 2-8° C.
